SEQUENCE LISTING (25 sequences)

SEQ ID NO: 1 - protein, 112 aa, Artificial sequence
  Heavy chain framework consensus sequence
QVQLVESGGGLVQPGGSLRLSCAASGFTFSSYAMHWVRQAPGKGLEWVSVISGDGSNTYYADSVKGRFTISRDNSKNTLYLQMNSLRAEDTAVYYCARGDYWGQGTLVTVSS

SEQ ID NO: 2 - protein, 115 aa, Artificial sequence
  anti-RAS intracellular scFv
QVQLVEAGGGLVQPGGSLRLSCAASGFSFSYLYMNWVRQAPGKGLEWVSYISSSGRTIYYADSVKGRFTISRDNAKNSLYLQMNSLRDEDTAVYYCARGRFFDYWGQGTLVTVSS

SEQ ID NO: 3 - protein, 115 aa, Artificial sequence
  anti-RAS intracellular scFv
QVQLVEAGGGLVQPGGSLRLSCAASGFSFSTFSMNWVRQAPGKGLEWVSYISSSGRTIYYADSVKGRFTISRDNAKNSLYLQMNSLRDEDTAVYYCARGRFFDYWGQGTLVTVSS

SEQ ID NO: 4 - protein, 116 aa, Artificial sequence
  anti-RAS intracellular scFv
EVQLLESGGGLVQPGGSLRLSCAASGFTFSSYAMSWVRQAPGKGLEWVSTISYGGSNTNYADSVKARFTISRDNSKNTLYLQMNSLRAEDTAVYYCAKGNNAFDYWGQGTLVTVSS

SEQ ID NO: 5 - protein, 126 aa, Artificial sequence
  anti-RAS intracellular scFv
EVQLVESGGGLVQPGGSLRLSCAASGFTFSSYAMSWVRQAPGKGLEWVSVISGKTDSTYYADSVKGRFTISRDNSKNTLYLQMNSLRAEDTAVYYCARGRGSLSGYYYYHYPFDYWGQGTLVTVSS

SEQ ID NO: 6 - protein, 115 aa, Artificial sequence
  anti-RAS intracellular scFv
EVQLLESGGGLVQPGGSLRLSCAASGFSFSYLYMNWVRQAPGKGLEWVSYISSSGRTIYYADSVKGRFTISRDNSKNTLYLQMNSLRAEDTAVYYCAKGRFFDYWGQGTLVTVSS

SEQ ID NO: 7 - protein, 115 aa, Artificial sequence
  anti-RAS intracellular scFv
EVQLLESGGGLVQPGGSLRLSCAASGFSFSYLYMNWVRQAPGKGLEWVSYISSSGRTIYYADSVKGRFTISRDNSKNTLYLQMNSLRAEDTAVYYCARGRFFDYWGQGTLVTVSS

SEQ ID NO: 8 - protein, 115 aa, Artificial sequence; identical to SEQ ID NO: 7
  anti-RAS intracellular scFv

SEQ ID NO: 9 - protein, 115 aa, Artificial sequence
  anti-RAS intracellular scFv
QVQLVESGGGLVQPGGSLRLSCAASGFSFSYLYMNWVRQAPGKGLEWVSYISSSGRTIYYADSVKGRFTISRDNSKNTLYLQMNSLRAEDTAVYYCARGRFFDYWGQGTLVTVSS

SEQ ID NO: 10 - protein, 115 aa, Artificial sequence
  anti-RAS intracellular scFv
EVQLLESGGGLVQPGGSLRLSSAASGFSFSYLYMNWVRQAPGKGLEWVSYISSSGRTIYYADSVKGRFTISRDNSKNTLYLQMNSLRAEDTAVYYSARGRFFDYWGQGTLVTVSS

SEQ ID NO: 11 - protein, 108 aa, Artificial sequence
  Light chain framework consensus sequence
DIVMTQSPSSLSASVGDRVTITCRASQSISYYLNWYQQKPGKAPKLLIYAASTLQSGVPSRFSGSGSGTDFTLTISSLQPEDFATYYCQQYYSTPRTFGQGTKVEIKR

SEQ ID NO: 12 - protein, 108 aa, Artificial sequence
  anti-RAS intracellular scFv
DIVMTQSPSFLSASVGDRVTITCRASQSISRYLNWYQQKPGKAPKLLIYAASSLQSGVPSRFSGSGSGTDFTLTISSLQPEDFAIYYCQQSYSTLLTFGGGTKLEIKR

SEQ ID NO: 13 - protein, 108 aa, Artificial sequence; identical to SEQ ID NO: 12
  anti-RAS intracellular scFv

SEQ ID NO: 14 - protein, 109 aa, Artificial sequence
  anti-RAS intracellular scFv
TDIQMTQSPSSLSASVGDRVTITCRASQSISSYLNWYQQKPGKAPKLLIYNASALQSGVPSRFSGSGSGTDFTLTISSLQPEDFATYYCQQYTSTPRTFGQGTKVEIKR

SEQ ID NO: 15 - protein, 112 aa, Artificial sequence
  anti-RAS intracellular scFv
DIQMTQSPSSLSASVGDRVTITCRASQSLVSISNYLAWYQQKPGKAPKLLIYAASSLESGVPSRFSGSGSGTDFTLTISSLQPEDFATYYCQQYNSLPQWTFGQGTKVEIKR

SEQ ID NO: 16 - protein, 109 aa, Artificial sequence
  anti-RAS intracellular scFv
TDIQMTQSPSSLSASVGDRVTITCRASQSISRYLNWYQQKPGKAPKLLIYAASSLQSGVPSRFSGSGSGTDFTLTISSLQPEDFATYYCQQSYSTLLTFGQGTKVEIKR

SEQ ID NO: 17 - protein, 109 aa, Artificial sequence; identical to SEQ ID NO: 16
  anti-RAS intracellular scFv

SEQ ID NO: 18 - protein, 109 aa, Artificial sequence; identical to SEQ ID NO: 14
  anti-RAS intracellular scFv

SEQ ID NO: 19 - protein, 108 aa, Artificial sequence
  anti-RAS intracellular scFv
DIVMTQSPSSLSASVGDRVTITCRASQSISSYLNWYQQKPGKAPKLLIYNASALQSGVPSRFSGSGSGTDFTLTISSLQPEDFATYYCQQYTSTPRTFGQGTKVEIKR

SEQ ID NO: 20 - protein, 109 aa, Artificial sequence; identical to SEQ ID NO: 16
  anti-RAS intracellular scFv

SEQ ID NO: 21 - protein, 15 aa, Artificial sequence
  Amino acid linker sequence
GGGGSGGGGSGGGGS

SEQ ID NO: 22 - protein, 9 aa, Human immunodeficiency virus type 1
RKKRRQRRR

SEQ ID NO: 23 - protein, 15 aa, Human immunodeficiency virus type 1
CGRKKRRQRRRPPQC

SEQ ID NO: 24 - protein, 16 aa, Drosophila melanogaster
RQIKIWFQNRRMKWKK

SEQ ID NO: 25 - protein, 9 aa, Artificial sequence
  FLAG-tag peptide
MDYKDDDDK

(19) United States
(12) Patent Application Publication
(10) Pub. No.: US 2005/0276800 A1
(43) Pub. Date: Dec. 15, 2005

(54) Intrabodies

(75) Inventors: Terrence Howard Rabbitts, Cambridge (GB); Tomoyuki Tanaka, Cambridge (GB)

Correspondence Address: PALMER & DODGE, LLP;KATHLEEN M. WILLIAMS, 111 HUNTINGTON AVENUE, BOSTON, MA, 02199, US

(73) Assignee: Medical Research Council

(21) Appl. No.: 11/127,932

(22) Filed: May 12, 2005

Related U.S. Application Data
(63) Continuation of application No. PCT/GB03/04943, filed on Nov. 14, 2003.

(30) Foreign Application Priority Data
Nov. 15, 2002 (GB) 0226727.6

(51) Int. Cl. G01N 33/53; A61K 39/395

(52) U.S. Cl. 424/133.1; 435/7.1

(57) ABSTRACT

The present invention related to antibodies that function within an intracellular environment. In particular the present invention relates to a particular antibody which the inventors have shown to bind to the oncogenic form of RAS. Uses of such an antibody are also described.

The present invention relates to antibodies that function within an intracellular environment. In particular the present invention relates to methods for generating antibodies which are soluble and are capable of specifically binding to antigen within an intracellular environment

Intracellular antibodies or intrabodies have been demonstrated to function in antigen recognition in the cells of higher organisms (reviewed in Cattaneo, A. & Biocca, S. (1997) Intracellular Antibodies: Development and Applications. Landes and Springer-Verlag). This interaction can influence the function of cellular proteins which have been successfully inhibited in the cytoplasm, the nucleus or in the secretory pathway. This efficacy has been demonstrated for viral resistance in plant biotechnology (Tavladoraki, P., et al. (1993) Nature 366: 469-472) and several applications have been reported of intracellular antibodies binding to HIV viral proteins Mhashilkar, A. M., et al. (1995) EMBO J. 14: 1542-51; Duan, L. & Pomerantz, R. J. (1994) Nucleic Acids Res 22: 5433-8; Maciejewski, J. P., et al. (1995) Nat Med 1: 667-73; Levy-Mintz, P., et al (1996) J. Virol. 70: 8821-8832) and to oncogene products (Biocca, S., Pierandrei-Amaldi, P. & Cattaneo, A. (1993) Biochem Biophys Res Commun 197: 422-7; Biocca, S., Pierandrei-Amaldi, P., Campioni, N. & Cattaneo, A. (1994) Biotechnology (NY) 12: 39 & 9; Cochet, O., et al. (1998) Cancer Res 58: 1170-6). The latter is an important area because enforced expression of oncogenes often occurs in tumour cells after chromosomal translocations (Rabbitts, T. H. (1994) Nature 372: 143-149). These proteins are therefore important intracellular therapeutic targets (Rabbitts, T. H. (1998) New Eng. J Med 338: 192-194) which could be inactivated by binding with intracellular antibodies. Finally, the international efforts at whole genome sequencing will produce massive numbers of potential gene sequences which encode proteins about which nothing is known. Functional genomics is an approach to ascertain the function of this plethora of proteins and the use of intracellular antibodies promises to be an important tool in this endeavour as a conceptually simple approach to knocking-out protein function directly by binding an antibody inside the cell. Simple approaches to derivation of antibodies which function in cells are therefore necessary if their use is to have any impact on the large number of protein targets. In normal circumstances, the biosynthesis of immunoglobulin occurs into the endoplasmic reticulum for secretion as antibody. However, when antibodies are expressed in the cell cytoplasm (where the redox conditions are unlike those found in the ER) folding and stability problems occur resulting in low expression levels and the limited half-life of antibody domains. These problems are most likely due to the reducing environment of the cell cytoplasm (Hwang, C., Sinskey, A. J. & Lodish, H. F. (1992) Science 257: 1496-502), which hinders the formation of the intrachain disulphide bond of the VH and VL domains (Biocca, S., Ruberti, F., Tafani, M., Pierandrei-Amaldi, P. & Cattaneo, A. (1995) Biotechnology (NY) 13: 1110-5; Martineau, P., Jones, P. & Winter, G. (1998) J Mol Biol 280: 117-127) important for the stability of the folded protein. However, some scFv have been shown to tolerate the absence of this bond (Proba, K, Honegger, A. & Pluckthun, A. (1997) J Mol Biol 265:161-72; Proba, K., Worn, A., Honegger, A. & Pluckthun, A. (1998) J Mol Biol 275: 245-53) which presumably depends on the particular primary sequence of the antibody variable regions. No rules or consistent predictions until the present invention, have been made about those antibodies which will tolerate the cell cytoplasm conditions. A further problem is the design of expression formats for intracellular antibodies and much effort has be expended on using scFv in which the VH and VL segments (i.e. the antibody combining site) are linked by a polypeptide linker at the C-terminus of VH and the N-terminus of V_{L} (Bird, R. E., et al. (1988) Science 242: 423-6). While this is the most successful form for intracellular expression, it has a drawback in the lowering of affinity when converting from complete antibody (e.g. from a monoclonal antibody) to a scFv. Thus not all monoclonal antibodies can be made as scFv and maintain function in cells. Finally, different scFv fragments have distinct properties of solubility or propensity to aggregate when expressed in this cellular environment.

Antibodies are used extensively in bioscience as in vitro tools for recognising target antigens and for medical applications such as diagnosis or therapeutics. Recently gene cloning technologies have allowed the genes for coding antibodies to be manipulated and expressed intracellularly (Cattaneo and Biocca, 1999a). Intracellular antibodies (ICAb) with specific and high-affinity binding properties have great potential for application in the therapy of human diseases in which target proteins or protein interactions are found only inside the target cell. A suitable form for ICAb expression is the single-chain antibody, also known as single chain variable fragment or scFv (Biocca et al., 1994; Cohen, 2002; Marasco et al., 1993), which is composed of the heavy and light-chain variable domains and a flexible linker peptide to fuse them (Bird et al., 1988; Huston et al., 1988).

Application of functional scFv as ICAbs have been exploited and achieved in several fields. There is potential for their use in cancer cells, where there occurs chromosomal translocations or somatic mutations effectively producing tumour-specific intracellular proteins (Rabbitts, 1994; Rabbitts and Stocks, 2002). As the protein products are inside in the cell, rather than exposed on the cell surface, conventional antibody therapy is not an option. The scFv format is suitable for intracellular use because of its optimal size and ease of expression from vectors since the VH and VL segments are present on a single macromolecule, and thus requiring no inter-chain disulphide linkage to hold together the two chains. Several such antibody fragments have been demonstrated to be effective in targeting proteins in vivo (Biocca et al., 1993; Rondon and Marasco, 1997; Tavladoraki et al., 1993), but there remain few antibodies which work effectively in intracellular reducing environment because there are often problems with correct folding and their resulting in lack of function, low expression and short half life (Cattaneo and Biocca, 1999b). Indeed, it has been generally found that most scFv which are derived from hybridomas do not function effectively in vivo, regardless of their having sufficient high affinity and antigen specificity. Furthermore, the intra-domain disulphide bond does not form in scFv expressed in the cytoplasm of eukaryotic cells bonds (Biocca et al., 1995) but some scFv have been shown to tolerate the absence of this bond (Proba et al., 1998; Worn and Pluckthun, 1998a). At this time, there is no general rule or prediction of the requirements for soluble and stable intracellular antibodies.

Several approaches have been adopted to solve this problem. These include the modification of the sequence of VH and VL domains utilising random mutation to replace the need for disulphide bonds to stabilise scFv with high intrinsic stability (Proba et al., 1998; Worn and Pluckthun, 1998b) or use of frameworks which empirically prove to be effective in vivo (Tse et al., 2002; Visintin et al., 2002).

However at the time of filing, there remains a need in the art to identify characteristics of intracellular antibodies which allow them to be soluble within an intracellular environment whilst maintaining the ability to interact specifically with one or more antigens/ligands. Such antibodies will have wide ranging prophylactic and/or therapeutic applications.

### SUMMARY OF THE INVENTION

The present inventors recently developed a selection method to isolate intracellular antibodies which primarily depends on their function inside yeast and mammalian cells, described as intracellular antibody capture (IAC) technology (Visintin et al., 1999) (WO00/54057). The inventors used such antibodies as a model to evaluate a consensus scaffold derived from IAC methods and described in PCT/GB02/003512.

The present inventors have isolated several anti-RAS scFv molecules. Surprisingly, they have found that poor solubility and stability exhibited by some of them in vivo could be improved by mutating particular framework region amino acids to other particular amino acids. Moreover, they discovered that some antibodies, for example I21 as shown in FIG. 3, are soluble within an intracellular environment, however exhibit low affinity for their specific ligand, whereas other antibodies exhibit high antigen binding affinity but low intracellular solubility. From these studies the inventors were able to determine some structural characteristics of intracellular antibodies which confer upon them the ability to be both soluble and interact specifically with one or more ligands/antigens within such an environment.

Importantly, they also discovered that the presence of an intradomain heavy chain variable domain disulphide bond is not required for an antibody of the invention to be soluble and interact specifically with one or more ligands within an intracellular environment. Moreover, antibodies comprising a single domain type only and which possess certain defined characteristics disclosed herein are soluble and are capable of interacting specifically with one or more ligands within such an environment.

Thus, in a first aspect the present invention provides a method for the generation of an antibody which is suitable for intracellular use comprising the steps of:
(a) testing two or more antibodies for their ability to bind to one or more antigens specifically within an intracellular environment, and selecting an antibody which is capable of binding to one or more antigens specifically within such an environment.
(b) testing two or more antibodies for their ability to be soluble within an intracellular environment, and selecting an antibody which is soluble within such an environment; and
(c) generating the antibody from the framework regions of the antibody selected in step (b) and the CDR sequences from the antibody selected in step (a).

As herein defined, the term ‘suitable for intracellular use’ means that those antibodies referred to above are intracellularly soluble and are capable of interacting with one or more antigens specifically via the CDRs comprising the antigen binding site. In the case of a heavy variable domain only antibody, for example a Dab, the relevant CDRs are the heavy chain variable domain CDRs. In the case of a heavy and light chain antibody the CDRs comprising the antigen binding site are those of the variable light chain domain and the variable heavy chain domain.

Antibodies generated according to the above method may comprise both light and heavy chain variable domains or a single domain type only (single domain type antibodies). Those skilled in the art will appreciate that this list is not intended to be exhaustive.

As referred to herein the term ‘a single variable domain type antibody’ means an antibody as herein defined which comprises either one or more heavy chain variable domains or one or more light chain domains but not both heavy and light chain variable domains. Advantageously, a single variable domain type antibody according to the invention is a Dab. As herein defined a ‘Dab’ is a single variable heavy chain domain or a single variable light chain domain optionally attached to a ‘bulking group’. The ‘bulking group’ as herein defined may comprise one or more antibody constant region domains. Alternatively, the ‘bulking group’ may comprise components of non-immunoglobulin origin. These may include cytotoxins, fluorescent or other forms of labels. For the avoidance of any doubt, a Dab as herein defined may comprise a light or heavy chain variable domain alone, that is in the absence of a bulking group. Those skilled in the art will appreciate that this list is not intended to be exhaustive.

Advantageously, antibodies generated using the method referred to above will comprise one or more of the framework region amino acid sequences selected from the group consisting of: Con (SEQ ID No: 1 (VH), and SEQ ID No: 11 (VL)); I21 (SEQ ID No: 4 (VH) and 14 (VL)); and I21R33 (SEQ ID No 7 (VH) and 17 (VL)). Or any of those heavy chain variable domain sequences in which amino acids 22 and 92 are not cysteine residues.

The inventors surprisingly found that the formation of a heavy variable domain intradomain disulphide bridge is not required in order to obtain an antibody which is both soluble within an intracellular environment. Moreover, they found that antibodies comprising only heavy chain variable domains and/or light chain variable domains only are capable of exhibiting solubility within such an environment.

Using these findings the inventors were able to devise a method for the de novo synthesis of an antibodies which are soluble within an intracellular environment whilst also being capable of interacting specifically with one or more antigens within such an environment.

Thus, in a further aspect the present invention provides a method for generating an antibody suitable for intracellular use comprising the step of synthesising the antibody using at least any of the framework sequences, (or the nucleic acid encoding them) selected from the list consisting of the following: the heavy chain framework region depicted in FIG. 3 as I21 and designated SEQ 4, the heavy chain framework region of the consensus sequence designated SEQ 1 and depicted as Con, the heavy chain framework region depicted in FIG. 3 as I21R33 and designated SEQ 7, the light chain framework region depicted in FIG. 3 as I21 and designated SEQ 14, the light chain framework region of the consensus sequence designated SEQ 11 and depicted as Con, the light chain framework region depicted in FIG. 3 as I21R33 and designated SEQ 17; or any of those heavy framework sequences wherein the amino acid residues at positions 22 and 92 (according to Kabat) are not cysteine residues.

According to the above aspects of the invention, the term ‘synthesising the antibody’ includes within its scope the selection of whole/intact antibodies comprising the sequences referred to above, and/or the selection of antibody fragments comprising the sequences referred to above and their subsequent assembly.

In addition the term ‘synthesising the antibody’ includes within its scope the synthesis of an antibody as described above comprising the step of assembling or synthesising nucleic acid encoding the various sequences or fragments thereof, referred to above. The synthesis of nucleic acid may include a PCR based approach. Those skilled in the art will be aware of other suitable methods for the synthesis of nucleic acid encoding the sequences referred to above.

Advantageously, antibodies generated according to this aspect of the invention are Dabs or scFVs. In an alternative embodiment of this aspect of the invention, the method is for generating an antibody comprising both heavy and light chains suitable for intracellular use. Advantageously antibodies generated using the above method are capable of interacting specifically with mutant RAS. In such a case the method further comprises the step of combining the relevant framework regions as shown in FIG. 3 with the corresponding CDR sequences as shown in FIG. 3. Preferably the method comprises synthesising the antibody from one or more of those sequences selected from the group designated I21R33, I21R33VHI21VL, Con 33 and I21R33 (VHC22S;C92S).

One skilled in the art will appreciate that antibodies according to the above aspect of the invention may be generated and subsequently the framework and/or one or more CDR sequences modified by mutation in order to modulate the in vivo solubility and/or specific antigen binding affinity of the resultant antibody. Advantageously, the modification involves substitution mutagenesis. Methods for performing mutagenesis involve standard laboratory techniques and will be familiar to those skilled in the art The inventors have also surprisingly found that the in vivo solubility of an antibody can be improved by mutating one or more variable domain framework amino acids comprising at least the heavy chain variable domain of the antibody into those framework amino acids exhibited by antibodies which the inventors have identified to be soluble within such an environment In particular, the inventors have found that mutation of scFv 33 (as shown in FIG. 3) of the 13 amino acid residues which are different between scFv 33 and scFv I21 to those amino acids exhibited by scFv I21 in those positions results in the conversion of an intracellularly insoluble molecule into one which is intracellularly soluble.

Thus is a further aspect, the present invention provides a method for converting an antibody molecule which comprises a heavy chain variable domain which falls within the VHIII subgroup of heavy chains, into one which is which is suitable for intracellular use comprising the step of mutating one or more framework amino acids (or the nucleic acid encoding them) comprising at least one of the antibodies heavy chain variable domains to those indicated (according to Kabat numbering) in the group consisting of the following: VH position 1, glutamine; VH position 5 lysine; VH position 7, serine; VH position 74, serine; VH position 77, threonine; VH position 84, VH alanine, VH position 22, any amino acid other than cysteine; VH position 92, any other amino acid other than cysteine.

Antibodies according to the present invention are advantageously, scFv or Dabs as herein defined. Preferably the Dabs are heavy chain variable domain Dabs.

In a further aspect still, the invention provides a method for converting an antibody molecule which comprises a light chain variable domain which falls within the VK1 subgroup of light chains, into one which is which is suitable for intracellular use comprising the step of mutating one or more framework amino acids (or the nucleic acid encoding them) comprising at least one of the antibodies light chain variable domains to those indicated (according to Kabat numbering) in the group consisting of the following: VL position 0, threonine; VL position 4, Glutamine.

Antibodies according to the present invention are advantageously, scFv or Dabs as herein defined. Preferably the Dabs are heavy chain variable domain Dabs.

In particular, the present inventors have found that the intracellular antibodies described by the sequences I21, I21R33 and I21R33 (VHC22S; C92S) as shown in FIG. 3 are suitable for intracellular use.

Thus, in a further aspect still, the present invention provides a method for converting an antibody molecule into one which is suitable for intracellular use comprising the step of mutating one or more framework amino acids (or the nucleic acid encoding it) comprising at least the antibodies heavy chain variable domain so that the resultant framework amino acid sequence comprises any of those sequences which are selected from the group consisting of the following as shown in FIG. 3: SEQ 1 (VH-Con), SEQ 4 (VH-I21), SEQ 7 (VH-I21R33) 17 or any of those heavy framework sequences wherein the amino acid residues at positions 22 and 92 (according to Kabat) are not cysteine residues.

In a preferred embodiment of the above aspect of the invention the method comprises the steps of: (a) Mutating the antibodies heavy chain variable domain (or the nucleic acid encoding it) so that the resultant framework amino acid sequence comprises any of those sequences which are selected from the group consisting of the following as shown in FIG. 3: SEQ 1 (VH-Con), SEQ 4 (VH-I21), SEQ 7 (VH-I21R33) or any of those heavy framework sequences wherein the amino acid residues at positions 22 and 92 (according to Kabat) are not cysteine residues; and (b) additionally mutating the antibodies light chain variable domain so that the resultant antibody framework amino acid sequence comprises any of those sequences which are selected from the group consisting of the following as shown in FIG. 3: SEQ 11 (VL-Con), SEQ 14 (VL-I21), SEQ 17 (VL-I21R33).

According to the above aspects of the invention, advantageously, the mutation step is performed at the nucleic acid level using methods familiar to those skilled in the art.

Intracellularly binding antibodies generated according to the above aspects of the invention may be single variable domain type antibodies (single variable domain antibodies), or they may comprise more than one variable domain type.

As referred to herein the term ‘a single variable domain type antibody’ means an antibody as herein defined which comprises either one or more heavy chain variable domains or one or more light chain domains but not both heavy and light chain variable domains. Advantageously, a single variable domain type antibody according to the invention is a Dab. As herein defined a ‘Dab’ is a single variable heavy chain domain or a single variable light chain domain optionally attached to a ‘bulking group’. The ‘bulking group’ as herein defined may comprise one or more antibody constant region domains. Alternatively, the ‘bulking group’ may comprise components of non-immunoglobulin origin. These may include cytotoxins, fluorescent or other forms of labels. Those skilled in the art will appreciate that this list is not intended to be exhaustive. Most advantageously, a ‘Dab’ according to the present invention comprises a single heavy chain variable domain attached to a bulking group as herein defined. For the avoidance of any doubt a Dab as herein defined may comprise a light chain variable domain or a heavy chain variable domain is isolation, that is in the absence of a bulking group.

According to the methods of the present invention, antibodies may comprise both light and heavy chain variable domains or either light or heavy chain variable domains but not both. More advantageously, the antibody comprises both light and heavy chain variable domains. Most advantageously, the antibody is an scFv.

In a further aspect still, the present invention provides an antibody molecule obtained using any of the methods of the invention.

In yet a further aspect, the present invention provides a library, wherein the library is generated from any one or more of the framework region sequences selected from the group shown in FIG. 3 consisting of: VH I21 (SEQ ID No: 4), VHI21R3 (SEQ ID No: 7), or either of those sequences wherein one or more of amino acids 22 and 92 are not cysteines; VLI21 (SEQ ID No: 14) and VLI21R33 (SEQ ID No:17).

The inventors consider that antibodies according to the invention will be of great therapeutic value.

Thus in a further aspect the present invention provides a composition comprising one or more antibodies according to the present invention and a pharmaceutically acceptable carrier, diluent or exipient.

In a further aspect still, the present invention provides the use of one or more antibodies according to the present invention in the preparation of a medicament for the prophylaxis and/or treatment of one or more disease/s.

In a final aspect the present invention provides the use of one or more antibodies according to the present invention in the preparation of a medicament for specifically interacting with one or more antigens within an intracellular environment

### DETAILED DESCRIPTION OF THE INVENTION

Definitions

Immunoglobulins molecules, according to the present invention, refer to any moieties which are capable of binding to a target. In particular, they include members of the immunoglobulin superfamily, a family of polypeptides which comprise the immunoglobulin fold characteristic of antibody molecules, which contains two beta sheets and, usually, a conserved disulphide bond. Members of the immunoglobulin superfamily are involved in many aspects of cellular and non-cellular interactions in vivo, including widespread roles in the immune system (for example, antibodies, T-cell receptor molecules and the like), involvement in cell adhesion (for example the ICAM molecules) and intracellular signalling (for example, receptor molecules, such as the PDGF receptor). The present invention relates to antibodies, particularly scFv or Dabs.

Antibodies as used herein, refers to complete antibodies or antibody fragments capable of binding to a selected target, and including Fv, ScFv, Fab′ and F(ab′)₂, Dab, monoclonal and polyclonal antibodies, engineered antibodies including chimeric, CDR-grafted and humanised antibodies, and artificially selected antibodies produced using phage display or alternative techniques. Small fragments, such as Fv and scFv, possess advantageous properties for diagnostic and therapeutic applications on account of their small size and consequent superior tissue distribution. Preferably, the antibody is a Dab or scFv.

As herein defined a ‘Dab’ is a single variable heavy chain domain or a single variable light chain domain optionally attached to a ‘bulking group’. The ‘bulking group’ as herein defined may comprise one or more antibody constant region domains. Alternatively, the ‘bulking group’ may comprise components of non-immunoglobulin origin. These may include cytotoxins, fluorescent or other forms of labels. For the avoidance of any doubt, a Dab as herein defined may comprise a light or heavy chain variable domain alone, that is, in the absence of a bulking group. Those skilled in the art will appreciate that this list is not intended to be exhaustive.

Heavy chain variable domain refers to that part of the heavy chain of an immunoglobulin molecule which forms part of the antigen binding site of that molecule. The VHIII subgroup describes a particular sub-group of heavy chain variable regions (the VHIII). Generally immunoglobulin molecules having a variable chain amino acid sequence falling within this group possess a VH amino acid sequence which can be described by the VHIII consensus sequence in the Kabat database.

Light-chain variable domain refers to that part of the light chain of an immunoglobulin molecule which forms part of the antigen binding site of that molecule. The VkI subgroup of immunoglobulin molecules describes a particular sub-group of variable light chains. Generally immunoglobulin molecules having a variable chain amino acid sequence falling within this group possess a VH amino acid sequence which can be described by the V_{K}I consensus sequence in the Kabat database.

Framework region of an immunoglobulin heavy and light chain variable domain. The variable domain of an immunoglobulin molecule has a particular 3 dimensional conformation characterised by the presence of an immunolgobulin fold. Certain amino acid residues present in the variable domain are responsible for maintaining this characteristic immunoglobulin domain core structure. These residues are known as framework residues and tend to be highly conserved.

CDR (complementarity determining region) of an immunoglobulin molecule heavy and light chain variable domain describes those amino acid residues which are not framework region residues and which are contained within the hypervariable loops of the variable regions. These hypervariable loops are directly involved with the interaction of the immunoglobulin with the ligand. Residues within these loops tend to show less degree of conservation than those in the framework region.

Intracellular means inside a cell, and the present invention is directed to those immunoglobulins which will bind to ligands/targets selectively within a cell (including the nucleus). The cell may be any cell, prokaryotic or eukaryotic, and is preferably selected from the group consisting of a bacterial cell, a yeast cell and a higher eukaryote cell. Most preferred are yeast cells and mammalian cells. As used herein, therefore, “intracellular” immunoglobulins and targets or ligands are immunoglobulins and targets/ligands which are present within a cell. In addition the term ‘Intracellular’ refers to environments which resemble or mimic an intracellular environment. Thus, “intracellular” may refer to an environment which is not within the cell, but is in vitro. For example, the method of the invention may be performed in an in vitro transcription and/or translation system, which may be obtained commercially, or derived from natural systems.

Consensus sequence of V_{H} and V_{L} chains in the context of the present invention refers to the consensus sequences of those V_{H} and V_{L} chains from immunoglobulin molecules which can bind selectively to a ligand in an intracellular environment. The residue which is most common in any one given position, when the sequences of those immunoglobulins which can bind intracellularly are compared is chosen as the consensus residue for that position. The consensus sequence is generated by comparing the residues for all the intracellularly binding immunoglobulins, at each position in turn, and then collating the data. In this case the sequences of 18 immunoglobulins was compared.

Specific (antibody) binding in the context of the present invention, means that the interaction between the antibody and the ligand are selective, that is, in the event that a number of molecules are presented to the antibody, the latter will only bind to one or a few of those molecules presented. Advantageously, the antibody-ligand interaction will be of high affinity. The interaction between immunoglobulin and ligand will be mediated by non-covalent interactions such as hydrogen bonding and Van der Waals forces.

The term ‘suitable for intracellular use’ means that those antibodies (suitable for intracellular use) are intracellularly soluble and are capable of interacting with one or more antigens specifically via the CDRs comprising the antigen binding site. In the case of a heavy variable domain only antibody, for example a Dab, the relevant CDRs are the heavy chain variable domain CDRs. In the case of a heavy and light chain antibody the CDRs comprising the antigen binding site are those of the variable light chain domain and the variable heavy chain domain.

The term ‘intracellularly functional antibody’ means that the antibody is of sufficient intracellular solubility so that the CDRs of the variable domains are capable of interacting specifically with their one or more antigens within an intracellular environment. Thus the term ‘intracellularly functional antibody’ as herein defined is synonymous with the term ‘suitable for intracellular use’.

A repertoire in the context of the present invention refers to a set of molecules generated by random, semi-random or directed variation of one or more template molecules, at the nucleic acid level, in order to provide a multiplicity of binding specificities. In this case the template molecule is one or more of the VH and/or VL domain sequences herein described. Methods for generating repertoires are well characterised in the art.

The term ‘library’ according to the present invention refers to a mixture of polypeptides or nucleic acids. The library is composed of members. Sequence differences between library members are responsible for the diversity present in the library. The library may take the form of a simple mixture of polypeptides or nucleic acids, or may be in the form organisms or cells, for example bacteria, viruses, animal or plant cells and the like, transformed with a library of nucleic acids. Typically, each individual organism or cell contains only one member of the library. In certain applications, each individual organism or cell may contain two or more members of the library. Advantageously, the nucleic acids are incorporated into expression vectors, in order to allow expression of the polypeptides encoded by the nucleic acids. In a preferred aspect, therefore, a library may take the form of a population of host organisms, each organism containing one or more copies of an expression vector containing a single member of the library in nucleic acid form which can be expressed to produce its corresponding polypeptide member. Preferably, the library encodes a repertoire of immunoglobulin molecules. A “repertoire” refers to a set of molecules generated by random, semi-random or directed variation of one or more template molecules, at the nucleic acid level, in order to provide a multiplicity of binding specificities. In this case the template molecule is one or more of the V_{H} and/or V_{L} amino acid sequences herein described. Methods for generating repertoires are well characterised in the art.

### BRIEF DESCRIPTION OF THE FIGURES

FIG. 1. Intracellular antibody capture of anti-RAS scFv.

2.7×10¹³ clones from three different phage libraries (de Wildt et al., 2000; Sheets et al., 1998) (total diversity 7.0×10⁹) were screened with purified HaRASG12V antigen in vitro. 1.18×10⁶ phage were recovered, phagemid DNA was prepared and scFv fragments cloned into the yeast vector pVP16 to make sub-library of 4.13×10⁶ clones. 8.45×10⁷ yeast clones were screened in yeast L40 strain expressing the LexA-RASG 12V bait 428 colonies grew on histidine selective plates and showed strong activation of the lacZ gene, determined by β-gal filter assay. All prey plasmids were isolated from histidine-independent and β-gal positive yeast colonies and were fingerprinted by digestion with restriction enzymes, BstN1, Msp1, Mbo1, RsaI or Hinf1 to identify the differing scFv clones. Subsequently 57 scFv clones which had different DNA fingerprinting patterns were re-tested in yeast with LexA-RASG12V bait and three scFv (which were originated from different libraries were isolated). Of these three anti-RAS scFv, only two detectably bound RAS protein in a mammalian reporter assay.

FIG. 2. Interaction of anti-RAS scFv with RAS protein in mammalian cells.

A. Luciferase Assay; COS7 cells were transiently co-transfected with various scFv-VP16 activation domain fusions and the GAL4-DBD bait plasmid pM1-HRASG12V (closed boxes) or pM1-lacZ (open boxes), together with the firefly luciferase reporter plasmid pG5-Luc and an internal Renilla luciferase control plasmid pRL-CMV. scFv-VP16 prey vectors were used expressing anti-RAS scFv33, J48 and I21 or anti- -gal scFvR4 (Martineau et al., 1998). The luciferase activities were measured 48 hours after transfection using Dual Luciferase Assay System (Promega) and a luminometer. The luciferase activities of each assay were normalised to the Renilla luciferase activity (used as internal control for the transfection efficiency). The fold luciferase induction level is shown with the activity of each scFv-VP16 with non-relevant bait taken as baseline.

B. In situ immunofluorescence study; COS7 cells were transiently co-transfected with pEF-myc-nuc-scFv J48 (anti-RAS scFv) or scFvR4 (anti- -gal scFv) and pHM6-RAS vectors expressing the RAS antigen. After 48 hours, cells were fixed and stained with 9E10 monoclonal antibody (detecting the myc tagged scFv) and rabbit anti-HA tag polyclonal serum, followed by secondary fluorescein conjugated anti-mouse and Cy3 conjugated anti-rabbit antibodies, respectively. The staining patterns were examined using a BioRadiance confocal microscope. Co-location of antigen and ICAb fluorescence was found for scFv J48 co-expressed with RAS.

Green (fluorescein)=fluorescence of scFv; Red (Cy3)=fluorescence of antigen

FIG. 3. Sequence of anti-RAS intracellular scFv.

The nucleotide sequences were obtained and the derived protein translations (shown as single letter code) were aligned. Dashes in framework (FR) represent identities with the consensus (CON) sequence (derived from anti-BCR and anti-ABL scFv isolated by the IAC method (Tse et al., 2002)). The numbers indicate the reference positions of the residues, according to the system by Lefranc et al (Lefranc and Lefranc, 2001) (top column number, indicated as IMGT) and Kabat et al (Kabat et al., 1991) (second column, Kabat). The 15 residues of the linker, (GGGGS)₃ between the heavy chain of variable domain (VH) and light chain (VL) are not shown. The complementarily determining regions (CDR) are highlighted on grey background and demarcated from framework regions (FR). Three anti-RAS intracellular scFv are designed as 33, J48 and I21. All anti-RAS scFv belong to the VH3 subgroup of heavy chain and V 1 subgroup of light chain shown in the middle (designed VH3 or V I) from the Kabat database (Kabat et al., 1991) or IGVH3 and IGVK1 from the Lefranc database (Lefranc and Lefranc, 2001). The mutated anti-RAS scFv are shown designed as I21K33, I21R33, I21R33VHI21VL, con33, and I21R33VH (C22SC92S). I21K33 comprises the six CDRs of scFv33 in the I21 framework and I21R33 is identical except for a mutation Lys94Arg; I21R33VH21VL comprises the VH domain of I21R33 fused to the VL domain of I21; con33 has all six CDRs of scFv33 in the canonical consensus framework (Tse et al., 2002); I21R33VH (C22S;C92S) is a mutant of clone I21R33 with the mutations CYS22SER and CYS92SER of the VH domain. There are only four amino acid differences (at positions H1, H5, L0, and L3) between consensus and I21R framework regions.

FIG. 4. Periplasmic expression and purification of anti-RAS scFv.

The scFv with pelB leader sequence at N-terminal and His6-tag and myc-tag at C-terminal were expressed periplasmically from the pHEN2-scFv vector in E. coli HB2151 using 1 mM IPTG for 2 hour at 30° C. in 1 litre of 2×TY medium including 100 μg/ml ampicilin and 0.1% glucose. After induction, the cells were harvested and extracted in 4 ml of ice cold 1×TES buffer (0.2 M Tris-HCl (pH 7.5), 0.5 mM EDTA, 0.5 M sucrose) and a further 6 ml of 1:5 TES buffer was added. The supernatants of cell extracts were used as the soluble periplasmic fraction. The his-tagged scFv were purified by immobilised Ni²⁺ ion chromatography and fractionated by 15% SDS-PAGE and proteins revealed by Coomassie blue staining. The approximate yields of purified anti-RAS scFv33 and J48 were less than 100 μg per 1 litre culture; scFvI21R33, I21R-33VHI21L and I21 more than 3 mg per litre; con33,1 mg per litre.

E=Complete periplasmic extracts and P=purified scFv; M=Mw markers

FIG. 5. Specific antigen binding and competition ELISA of anti-RAS scFv.

Purified HRASG12V-GppNp (4 μg/ml, approximately 200 nM; black boxes) or bovine serum albumin (BSA, 30 mg/ml, approximately 450 μM; grey boxes) were coated on to ELISA plates for 1.5 hours at room temperature. For both sets of wells, 3% BSA in PBS was added for blocking and subsequently purified scFv (450 ng per well) was added and incubated overnight at 4° C. After washing with PBS-0.1% Tween 20, bound scFv was detected with HRP-conjugated anti-poly-histidine antibody (HIS-1, Sigma) and signals quantitated using Emax microplate reader (Molecular Devices). For competition assays (indicated in figure as +), scFv were pre-incubated with HRASG12V-GppNp (8 μg/ml; approx. 400 nM) for 30 min at room temperature before addition to ELISA well.

FIG. 6. Affinity measurements of anti-RAS scFv using BIAcore.

Biosensor measuremenst were made using the BIAcore 2000. Purified scFv from bacterial cultures were used.

A. Sensograms showing the binding of anti-RAS scFv with HRASG12V-GppNp antigen (immobilised 1500 RU). An injection volumes of 40 μl and flow rates of 20 μl/min were used. The purified scFv (10-2000 nM) were loaded on 2 channels of the chip, containing either immobilised HRASG12V-GppNp or no antigen. The sensograms of each measurement were normalised by the resonance of the channel without antigen.

B. The table summarises the value of association rate (Kon) and the dissociation rate (Koff) and calculated equilibrium dissociation constants (Kd) by BIAevaluation 2.1 software.

FIG. 7. Influence of framework residues on the solubility of expressed scFv in COS7 cells.

COS7 cells were transiently transfected with pEF-myc-cyto-scFv expression clones as indicated. Soluble and insoluble proteins were extracted, as described in materials and methods, and fractionated on 15% SDS-PAGE. After electrophoresis, protein were transferred to membranes and incubated with the anti-myc tag monoclonal antibody 9E10. The migration molecular weight markers (in kDa) are shown on the left. Arrows on the right indicate to the scFv fragment band.

FIG. 8. Improvement of intracellular interaction between anti-RAS ICAbs and RAS antigen by the mutation of framework sequences.

Mammalian two-hybrid antibody-antigen interaction assays were performed in COS7 cells.

A. COS7 were transfected with the pEFBOSVP16-scFv vectors and the pM1-RASG12V, together with the luciferase reporter clones and luciferase levels were determined as described in methods. The upper panel represents normalised fold induction of luciferase signals (zero being taken as signal from prey plasmid without scFv) for scFv-VP16 binding RAS antigen bait. The lower panel shows a Western blot of COS7 cell extracts after the expression of scFv-VP16 fusion proteins. ScFv-VP16 fusion proteins were detected by Western-blot using anti-VP16 (Santa Cruz Biotechnology, 14-5) monoclonal antibody and horseradish peroxidase (HRP)-conjugated anti-mouse IgG antibody.

ICAb scFv used as a control was anti- -gal R4 (Martineau et al., 1998). scFv33 mutants were (using Kabat et al (Kabat et al., 1991) and number in parenthesis also indicate numbering by Lefranc et al (Lefranc and Lefranc, 2001)) (see FIG. 3)

VH(A74S+S77T): substitutions Ala74(83)Ser and Ser77(86)Thr of VH

VH(84A): substitution Asp84(96)Ala of VH VH(D94K): substitution Arg94(106)Lys of VH

VL(0T+V3Q): addition Thr between linker and VL domain plus substitution

Val3(3)Gln of VL

VL(F10S): substitution Phe10(10)Ser of VL

VL(184T): substitution Ile84(100)Thr of VL

VH(Q1E+V5L+A7S+S28T)+VL(G100Q+V104L): substitutions Gln1(1)Glu, Val5(5)Leu, Ala7(7)Ser, Ser28(29)Thr of VH plus Gly100Gln and Val104Leu of VL.

B. COS7 cell two-hybrid antibody-antigen interaction assay using scFv with framework mutations to convert to consensus sequence scaffolds. The various scFv-VP16 prey constructs shown were transiently transfected with GAL4-RASG12V bait plasmid in COS7 cells and the luciferase activities were measured 48 hour after transfection. The fold luciferase activity level are shown in histogram with the activity of no scFv (prey plasmid without scFv) as baseline. The expression levels of scFv-VP16 in soluble fraction of COS7 cells are shown in lower panel. The bands were visualised by Western-blot using anti-VP16 (14-5) antibody and HRP-conjugated anti mouse IgG antibody.

FIG. 9. Inhibition of RAS-dependent NIH3T3 cells transformation activity by anti-RAS scFv.

Mutant HRAS G12V cDNA were subcloned into the mammalian expression vector pZIPneoSV(X) and anti-RAS scFv into pEF-FLAG-Memb vector which has plasma membrane targeting signal at C-terminal of scFv and FLAG-tag at N-terminal to scFv. 100 ng of pZIPneoSV(X)-RASG12V and 2 μg of pEF-FLAG-Memb-scFv were co-transfected into NIH 3T3 cells cloneD4. Two days later, the cells were transferred to 10 cm plates and grown for 14 days in DME medium containing 5% donor calf serum and penicillin and streptomycin. Finally, the plates were stained with crystal violet and foci of transformed cells were counted.

(A) Representative photograph of stained plates. Empty vector in left-top panel indicates co-transfection of pZIPneoSV(X) without RASG12V and pEF-FLAG-Memb without scFv as negative control. No foci formation was observed. The right-top panel indicates pZIPneoSV(X) with RASG12V pEF-FLAG-Memb without scFv as positive control. In the other plates, the RASG12V vector was co-transfected with either pEF-memb-scFvI21 or pEF-memb-scFvI21R33.

(B) Relative percentage of transformation foci was determined as a number of foci normalised to the focus formation induced by pZIPneoSV(X)HRASG12V and pEF-memb empty vector, which was set at 100. Results shown represent one experiment with each transfection performed in duplicate. Two additional experiments yielded similar results.

### GENERAL TECHNIQUES

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc. which are incorporated herein by reference) and chemical methods. In addition Harlow & Lane., A Laboratory Manual Cold Spring Harbor, N.Y., is referred to for standard Immunological Techniques.

De novo Synthesis of Antibodies

The present inventors have identified variable heavy chain and variable light chain framework residues which determine the in vivo solubility of antibodies comprising them.

Thus the invention provides a provides a method for the generation of an antibody suitable for intracellular use within an comprising the steps of:
(a) testing two or more antibodies for their ability to bind to one or more antigens specifically within an intracellular environment, and selecting an antibody which is capable of binding to one or more antigens specifically within such an environment.
(b) testing two or more antibodies for their ability to be soluble within an intracellular environment, and selecting an antibody which is soluble within such an environment; and
(c) generating the antibody from the framework regions of the antibody selected in step (b) and the CDR sequences from the antibody selected in step (a).

The present inventors have shown that the framework regions of depicted as I21 in FIG. 3 and designated SEQ ID No: 4 and 14 and the framework regions of the consensus designated SEQ ID No: 1 and 11 in FIG. 3, and SEQ ID No: 7 and 17 in FIG. 3 confer upon the an antibody comprising them the solubility required to function within an intracellular environment

In addition the above listed sequences which do not comprise cysteine residues at one or more of positions 22 and 92 according to Kabat (for VH) may also be used to confer upon the an antibody comprising them the solubility required to function within an intracellular environment. In particular those amino acid sequences wherein the cysteine at one or both positions is replaced by serine may be used to confer upon the an antibody comprising them the conformational stability and solubility required to function within an intracellular environment

Additionally, experiments have shown that those CDRs present on scFv molecules J48 and 33, and designated SEQ 2 and 12 (J48) and SEQ 3 and 13 (33) in FIG. 3 respectively confer upon an intracellularly stable antibody comprising them the ability to bind mutant RAS specifically.

Thus, any combination of the framework sequences above with those CDR sequences listed above when combined can be used to generate de novo a Dab or a light and heavy chain antibody which can specifically bind to mutant RAS within an intracellular environment.

Thus, it is reasonable to expect that ICAb libraries can be constructed which are based on the consensus ICAb scaffold and of sufficient diversity to allow primary screening directly in yeast cell assays, without recourse to a preliminary in vitro phage antibody screen with protein antigen. This would provide very clear technical advantages for example that antigens would not need to be expressed in vitro to provide for protein and the requirement for the yeast bait expression for IAC selection is merely DNA sequence. This opens the possibility of using IAC technology to select ICAbs which bind to any protein predicted from genome sequence analysis of any organism e.g. from the human genome sequencing programme.

A key question is the degree of library diversity required to contain effective intracellular antibodies and thus whether sufficient antibody diversity can be made and screened in yeast. The success of the current IAC requires to start with in vitro phage Ab screening of highly diverse libraries. However, such phage antibodies do not necessarily work as intracellular antibody without modification (because of solubility and folding problems in reducing environment) meaning that the effective diversity of these phage scFv libraries, as intracellular antibody libraries, is likely to be less than expected. The construction of specially designed human intracellular antibody libraries using randomised CDRs on the fixed consensus framework should increase effective ICAb diversity. This will make it possible to screen the library for ICAbs without the need for preliminary phage panning step, while keeping full effective diversity and accelerating the screening for intracellular antibodies.

The IAC Method for the Isolation of Antibodies

The IAC approach has several advantages compared with other screening methods. It is based on the yeast two-hybrid in vivo assay, which works as direct cytoplasmic selection of scFv. In addition, it theoretically allows the selection of antibody fragments (in the experiments described here scFv) against any cytoplasmically expressed antigen, including post-transcriptionally-modified proteins or especially protein complexes, as it allows targeting of antigen in its native form. A further consideration is that the screening process involves verification of candidate intracellular scFv in mammalian cells. By adopting these different bait and reporter systems, false-positive scFv are eliminated. In addition, the mammalian antigen-antibody interaction assay is performed at 37° C., compared with 30° C. in yeast or at room temperature (or at 4° C.) for in vitro phage screen. This step from yeast to mammalian cells makes it possible to select more thermal-tolerant intracellular scFv and because the isolation involves a mammalian assay, higher affinity interactions may be selected which are suitable for competitive binding of the target antigen with endogenous dimerisation molecules.

In this work, the present inventors have applied IAC technology against the oncogenic protein RAS and have isolated specific anti-RAS scFv which bind to this antigen in the cell cytoplasm. Sequence analysis demonstrates that all anti-RAS scFv belong to VH3 and V_{K}1 subgroup defined from Kabat database (Kabat et al., 1991) or IGHV3 and IGK1 subgroup defined from IMGT database (Lefranc and Lefranc, 2001). Most ICAb scFv selected which bind the antigens BCR or ABL (Tse et al., 2002) and TAU (Visintin et al., 2002), also belong to same subgroup, supporting the notion these subgroups of VH and VL framework can function intracellularly. This observation allowed a consensus framework scaffold to be defined (Tse et al., 2002).

The most important requirements for intracellular antibodies as therapeutic or bioscience research tools is that these antibodies (or antibody fragments) exhibit high stability, good expression levels and are functional within any compartment of mammalian cells. These are severe limitations and few scFv fragments derived from hybridomas are stable under a reducing environment without modification, even if they have good affinity in vitro. The intracellular antibody capture (IAC) technology described here, and previously (Tse et al., 2002; Visintin et al., 2002) overcome these difficulties being based on an in vivo genetic screen for the direct isolation of functional scFv.

Screening of Dab Libraries

The IAC method can equally be applied to the selection of single variable domain only antibodies (Dabs). An brief description of a suitable method is provided below:
The screening of synthetic Dab libraries may be performed according to the protocol of intracellular antibody capture (IAC) technology as described REF (see also a link within the Laboratory of Molecular Biology website http://mrc-lmb.cam.ac.uk) but excluding the phage panning step. In outline, 500 μg of pBTM-antigen and 1 mg of pVP16-Dab library 1 or pVP16-Dab library 2 are co-transfected into S. cerevisiae L40. Positive clones are selected by using auxotrophic markers, Trp, Leu and His. Positive colonies are selected for His prototropy and confirmed by β-galactosidase (β-gal) activity by filter assay. For the selected individual clones, false positive clones are eliminated and true positive clones are confirmed by re-testing of His independent growth and β-gal activation, using relevant and non-relevant bait vectors.
   Some Intracellular Antibodies Selected Using IAC Exhibit High Intracellular Solubility and Others Exhibit Low Intracellular Solubility

For example, using IAC screening for anti-RAS scFv, one scFv (I21) shows high yields in bacteria periplasm, high solubility in mammalian cells but poor affinity of interaction in mammalian cells whilst other scFv (scFv33 and J48) have high affinity but relatively low yields of soluble expressed protein. The I21 scFv framework sequence however conforms closely with the consensus framework (Tse et al., 2002) shown in FIG. 3, in both the VH and VL regions. Therefore, these results indicate that the consensus framework identified in FIG. 3 as SEQ 1 and 11 and also the I21 framework sequence shown in FIG. 3 and identified as SEQ 4 and 14, and also the consensus framework identified in FIG. 3 as I21R33 and designated SEQ 7 and 17 makes an ideal framework sequence for the generation of bespoke intracellular antibodies.

Methods for measuring the intracellular solubility of antibody molecules Methods for measuring the intracellular solubility of antibodies according to the present invention will be familiar to those skilled in the art. A suitable method involves the analysis of intracellular protein expression levels using, for example Western blot is analysis. A suitable protocol is outlined below. Those skilled in the art will be aware of other suitable methods:
To evaluate the expression level and solubility of scFv in mammalian cells, the scFv or scFv-VP16 fusion proteins were expressed in COS7 cells. For scFv expression, scFv DNA fragments were cloned into Nco1/Not1 sites of pEF-myc-cyto expression vector (Invitrogen). The day before transfection, COS7 cells were seeded at about 2×10⁵ per well in 6-well culture plate (Nunc). 1 μg of pEF-myc-cyto-scFv or pEF-BOS-scFv-VP16 were transiently transfected with 8 μl of LipofectAMINE. 48 hours after transfection, the cells were washed once with PBS, lysed for 30 minutes in ice cold extraction buffer (10 mM HEPES, pH 7.6, 250 mM NaCl, 5 mM EDTA, 0.5% NP-40, 1 μg/ml leupeptin, 1 μg/ml pepstatin A, 0.1 mg/ml aprotinin, 1 mM phenylmethanesulsonyl fluoride (PMSF)) and centrifuged for 10 minutes at 13,000 rpm at 4° C. The pellets (“insoluble” fraction) and the supernatants (“soluble” fraction) were analysed by SDS-PAGE, followed by Western blot using anti-myc (9E10) monoclonal antibody (for detection of scFv) or anti-VP16 (14-5, Santa-Cruz) monoclonal antibody (for scFv-VP16AD fusion) as primary antibody and HRP-conjugated rabbit anti-mouse IgG antibody (APB) as secondary antibody. The blots were visualised by enhanced chemiluminescence (ECL) detection kit (ABP)
   Antibodies Obtained According to the Methods of the Invention

Sequence analysis demonstrates that all anti-RAS scFv belong to VH3 and VK1 subgroup defined from Kabat database (Kabat et al., 1991) or IGHV3 and IGK1 subgroup defined from IMGT database (Lefranc and Lefranc, 2001). Most ICAb scFv selected which bind the antigens BCR or ABL (Tse et al., 2002) and TAU (Visintin et al., 2002), also belong to same subgroup, supporting the notion these subgroups of VH and V_{K} framework can function intracellularly. This observation allowed a consensus framework scaffold to be defined (Tse et al., 2002). In this screening for anti-RAS scFv, one scFv (I21) shows high yields in bacteria periplasm, high solubility in mammalian cells but poor affinity of interaction in mammalian cells whilst other scFv (scFv33 and J48) have high affinity but relatively low yields of soluble expressed protein. The I21 scFv framework sequence however conforms closely with the consensus framework (Tse et al., 2002) in both the VH and VL regions and in support of the utility of this consensus, the present inventors found that mutation of scFv33 to the consensus framework (con33) or to the I21 framework (I21R33) improved this function including solubility and binding. Finally, when the scFv33 was mutated to the I21R consensus framework, retaining the scFv33 CDR sequences, the ICAbs were able to perform the crucial biological function of inhibiting oncogenic RASG12V transformation of NIH3T3 cells. This is presumably due to the interaction of ICAb with the RAS target antigen (see FIG. 2). This illustrates the versatility of our approach in generating effective ICAbs for mammalian cell use.

the Requirement of Intracellularly Soluble Antibodies Comprising Both Light and Heavy Chains for Intradomain Disulphide Bridge Formation

The inventors have found that in order for antibodies to be intracellularly soluble then intradomain disulphide formation is not required. That is an antibody molecule comprising both light and heavy chains wherein at least one of the cysteine residues which are normally present at positions 22 and 92 according to the Kabat nomenclature is no longer present or has undergone mutation to another residue, for example serine, is capable of binding specifically to mutant RAS within an intracellular environment.

Advantageously, an antibody according to the invention comprises both light and heavy chains wherein the framework region of the antibody is that represented by SEQ 10 and SEQ 20 or the framework region of the antibody is the same as that represented by SEQ 10 and 20 except that one or more of residues 22 and 92 are not cysteine residues (according to Kabat numbering).

(a) Single Domain Type Antibodies According to the Invention.

The present inventors have found that antibodies comprising either heavy chain variable domains or light chain variable domains but not both (referred to as herein as single domain type only antibodies) are soluble within an intracellular environment whilst still being capable of interacting specifically with one or more ligands within such an environment.

Thus, antibodies comprising heavy chain variable domains and not light chain variable domains wherein the framework region sequences of the heavy chain variable domain are selected from the group shown in FIG. 3 and designated I21 (SEQ 4) and I21R33 (SEQ 7) are soluble within such an environment.

According to the above aspect of the invention preferably the single variable domain only antibody is one comprising a single heavy chain variable domain (heavy variable domain Dab).

Moreover, the inventors have found that antibodies comprising light chain variable domains and not heavy chain variable domains wherein the framework region sequences of the light chain variable domain are selected from the group shown in FIG. 3 and designated I21 (SEQ 14) and I21 R33 (SEQ 17) are soluble within such an environment and are capable of interacting specifically with their one or more ligands within such an environment.

Single domain antibodies may be prepared by any suitable technique. The preparation of single domain antibodies is described in detail in Ward et al., (1989) Nature 341: 544-546 and in European Patent Application 0 368 684 (Medical Research Council).

The Requirement of Antibodies Comprising Heavy Chain Variable Domains Only According to the Invention for Intradomain Disulphide Bridge Formation

The inventors have found that an antibody molecule according to the invention comprising one or more heavy chain variable domain only wherein at least one of the cysteine residues which are normally present at positions 22 and 92 in that domain according to the Kabat nomenclature is no longer present or has undergone mutation to another residue, for example serine, is capable of binding specifically to mutant RAS within an intracellular environment.

According to the above aspect of the invention preferably the single variable domain antibody is one comprising a single heavy chain variable domain (single heavy domain antibody).

Delivery of Antibodies According to the Present Invention to Cells

In order to introduce antibodies according to the present invention into an intracellular environment, cells are advantageously transfected with nucleic acids which encode the antibodies.

Nucleic acids encoding antibodies can be incorporated into vectors for expression. As used herein, vector (or plasmid) refers to discrete elements that are used to introduce heterologous DNA into cells for expression thereof. Selection and use of such vehicles are well within the skill of the artisan. Many vectors are available, and selection of appropriate vector will depend on the intended use of the vector, the size of the nucleic acid to be inserted into the vector, and the host cell to be transformed with the vector. Each vector contains various components depending on its function and the host cell for which it is compatible. The vector components generally include, but are not limited to, one or more of the following: an origin of replication, one or more marker genes, an enhancer element, a promoter, a transcription termination sequence and a signal sequence.

Moreover, nucleic acids encoding the antibodies according to the invention may be incorporated into cloning vectors, for general manipulation and nucleic acid amplification purposes.

Both expression and cloning vectors generally contain nucleic acid sequence that enable the vector to replicate in one or more selected host cells. Typically in cloning vectors, this sequence is one that enables the vector to replicate independently of the host chromosomal DNA, and includes origins of replication or autonomously replicating sequences. Such sequences are well known for a variety of bacteria, yeast and viruses. The origin of replication from the plasmid pBR322 is suitable for most Gram-negative bacteria, the 2m plasmid origin is suitable for yeast, and various viral origins (e.g. SV 40, polyoma, adenovirus) are useful for cloning vectors in mammalian cells. Generally, the origin of replication component is not needed for mammalian expression vectors unless these are used in mammalian cells competent for high level DNA replication, such as COS cells.

Most expression vectors are shuttle vectors, i.e. they are capable of replication in at least one class of organisms but can be transfected into another class of organisms for expression. For example, a vector is cloned in E. coli and then the same vector is transfected into yeast or mammalian cells even though it is not capable of replicating independently of the host cell chromosome. DNA may also be replicated by insertion into the host genome. However, the recovery of genomic DNA is more complex than that of exogenously replicated vector because restriction enzyme digestion is required to excise the nucleic acid. DNA can be amplified by PCR and be directly transfected into the host cells without any replication component.

Advantageously, an expression and cloning vector may contain a selection gene also referred to as selectable marker. This gene encodes a protein necessary for the survival or growth of transformed host cells grown in a selective culture medium. Host cells not transformed with the vector containing the selection gene will not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available from complex media.

As to a selective gene marker appropriate for yeast, any marker gene can be used which facilitates the selection for transformants due to the phenotypic expression of the marker gene. Suitable markers for yeast are, for example, those conferring resistance to antibiotics G418, hygromycin or bleomycin, or provide for prototrophy in an auxotrophic yeast mutant, for example the URA3, LEU2, LYS2, TRP1, or HIS3 gene.

Since the replication of vectors is conveniently done in E. coli, an E. coli genetic marker and an E. coli origin of replication are advantageously included. These can be obtained from E. coli plasmids, such as pBR322, Bluescript© vector or a pUC plasmid, e.g. pUC18 or pUC19, which contain both an E. coli replication origin and an E. coli genetic marker conferring resistance to antibiotics, such as ampicillin.

Suitable selectable markers for mammalian cells are those that enable the identification of cells expressing the desired nucleic acid, such as dihydrofolate reductase (DHFR, methotrexate resistance), thymidine kinase, or genes conferring resistance to G418 or hygromycin. The mammalian cell transformants are placed under selection pressure which only those transformants which have taken up and are expressing the marker are uniquely adapted to survive. In the case of a DHFR or glutamine synthase (GS) marker, selection pressure can be imposed by culturing the transformants under conditions in which the pressure is progressively increased, thereby leading to amplification (at its chromosomal integration site) of both the selection gene and the linked nucleic acid. Amplification is the process by which genes in greater demand for the production of a protein critical for growth, together with closely associated genes which may encode a desired protein, are reiterated in tandem within the chromosomes of recombinant cells. Increased quantities of desired protein are usually synthesised from thus amplified DNA.

Expression and cloning vectors usually contain a promoter that is recognised by the host organism and is operably linked to the desired nucleic acid. Such a promoter may be inducible or constitutive. The promoters are operably linked to the nucleic acid by removing the promoter from the source DNA and inserting the isolated promoter sequence into the vector. Both the native promoter sequence and many heterologous promoters may be used to direct amplification and/or expression of nucleic acid encoding the antibody. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Promoters suitable for use with prokaryotic hosts include, for example, the lactamase and lactose promoter systems, alkaline phosphatase, the tryptophan (trp) promoter system and hybrid promoters such as the tac promoter. Their nucleotide sequences have been published, thereby enabling the skilled worker operably to ligate them a desired nucleic acid, using linkers or adaptors to supply any required restriction sites. Promoters for use in bacterial systems will also generally contain a Shine-Delgarno sequence operably linked to the nucleic acid.

Preferred expression vectors are bacterial expression vectors which comprise a promoter of a bacteriophage such as phagex or T7 which is capable of functioning in the bacteria. In one of the most widely used expression systems, the nucleic acid encoding the fusion protein may be transcribed from the vector by T7 RNA polymerase (Studier et al, Methods in Enzymol. 185; 60-89, 1990). In the E. coli BL21( )E3) host strain, used in conjunction with pET vectors, the T7 RNA polymerase is produced from the lysogen DE3 in the host bacterium, and its expression is under the control of the IPTG inducible lac UV5 promoter. This system has been employed successfully for over-production of many proteins. Alternatively the polymerase gene may be introduced on a lambda phage by infection with an int-phage such as the CE6 phage which is commercially available (Novagen, Madison, USA). other vectors include vectors containing the lambda PL promoter such as PLEX (Invitrogen, NL), vectors containing the trc promoters such as pTrcHisXpress™ (Invitrogen) or pTrc99 (Pharmacia Biotech, SE), or vectors containing the tac promoter such as pKK223-3 (Pharmacia Biotech) or PMAL (new England Biolabs, MA, USA).

Suitable promoting sequences for use with yeast hosts may be regulated or constitutive and are preferably derived from a highly expressed yeast gene, especially a Saccharomyces cerevisiae gene. Thus, the promoter of the TRP1 gene, the ADHI or ADHII gene, the acid phosphatase (PH05) gene, a promoter of the yeast mating pheromone genes coding for the a- or α-factor or a promoter derived from a gene encoding a glycolytic enzyme such as the promoter of the enolase, glyceraldehyde-3-phosphate dehydrogenase (GAP), 3-phospho glycerate kinase (PGK), hexokinase, pyruvate decarboxylase, phosphofructokinase, glucose-6-phosphate isomerase, 3-phosphoglycerate mutase, pyruvate kinase, triose phosphate isomerase, phosphoglucose isomerase or glucokinase genes, the S. cerevisiae GAL 4 gene, the S. pombe nmt 1 gene or a promoter from the TATA binding protein (IBP) gene can be used. Furthermore, it is possible to use hybrid promoters comprising upstream activation sequences (UAS) of one yeast gene and downstream promoter elements including a functional TATA box of another yeast gene, for example a hybrid promoter including the UAS(s) of the yeast PH05 gene and downstream promoter elements including a functional TATA box of the yeast GAP gene (PH05-GAP hybrid promoter). A suitable constitutive PHO5 promoter is e.g. a shortened acid phosphatase PH05 promoter devoid of the upstream regulatory elements (UAS) such as the PH05 (−173) promoter element starting at nucleotide −173 and ending at nucleotide −9 of the PH05 gene.

Gene transcription from vectors in mammalian hosts may be controlled by promoters derived from the genomes of viruses such as polyoma virus, adenovirus, fowlpox virus, bovine papilloma virus, avian sarcoma virus, cytomegalovirus (CMV), a retrovirus and Simian Virus 40 (SV40), from heterologous mammalian promoters such as the actin promoter or a very strong promoter, e.g. a ribosomal protein promoter, and from promoters normally associated with immunoglobulin sequences.

Transcription of a nucleic acid by higher eukaryotes may be increased by inserting an enhancer sequence into the vector. Enhancers are relatively orientation and position independent. Many enhancer sequences are known from mammalian genes (e.g. elastase and globin). However, typically one will employ an enhancer from a eukaryotic cell virus. Examples include the SV40 enhancer on the late side of the replication origin (bp 100-270) and the CMV early promoter enhancer. The enhancer may be spliced into the vector at a position 5′ or 3′ to the desired nucleic acid, but is preferably located at a site 5′ from the promoter.

Advantageously, a eukaryotic expression vector may comprise a locus control region (LCR). LCRs are capable of directing high-level integration site independent expression of transgenes integrated into host cell chromatin, which is of importance especially where the gene is to be expressed in the context of a permanently-transfected eukaryotic cell line in which chromosomal integration of the vector has occurred.

Eukaryotic expression vectors will also contain sequences necessary for the termination of transcription and for stabilising the mRNA. Such sequences are commonly available from the 5′ and 3′ untranslated regions of eukaryotic or viral DNAs or cDNAs. These regions contain nucleotide segments transcribed as polyadenylated fragments in the untranslated portion of the mRNA encoding the immunoglobulin or the target.

Particularly useful for practising the present invention are expression vectors that provide for the transient expression of nucleic acids in mammalian cells. Transient expression usually involves the use of an expression vector that is able to replicate efficiently in a host cell, such that the host cell accumulates many copies of the expression vector, and, in turn, synthesises high levels of the desired gene product.

Construction of vectors according to the invention may employ conventional ligation techniques. Isolated plasmids or DNA fragments are cleaved, tailored, and religated in the form desired to generate the plasmids required. If desired, analysis to confirm correct sequences in the constructed plasmids is performed in a known fashion. Suitable methods for constructing expression vectors, preparing in vitro transcripts, introducing DNA into host cells, and performing analyses for assessing gene product expression and function are known to those skilled in the art. Gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe which may be based on a sequence provided herein. Those skilled in the art will readily envisage how these methods may be modified, if desired.

Antibodies may be directly introduced to the cell by microinjection, or delivery using vesicles such as liposomes which are capable of fusing with the cell membrane. Viral fusogenic peptides are advantageously used to promote membrane fusion and delivery to the cytoplasm of the cell.

Preferably, the immunoglobulin is fused or conjugated to a domain or sequence from such a protein responsible for translocational activity. Preferred translocation domains and sequences include domains and sequences from the HIV-1-trans-activating protein (Tat), Drosophila Antennapedia homeodomain protein and the herpes simplex-1 virus VP22 protein. By this means, the immunoglobulin is able to enter the cell or its nucleus when introduced in the vicinity of the cell.

Exogenously added HIV-1-trans-activating protein (Tat) can translocate through the plasma membrane and to reach the nucleus to transactivate the viral genome. Translocational activity has been identified in amino acids 37-72 (Fawell et al., 1994, Proc. Natl. Acad. Sci. U.S.A. 91, 664-668), 37-62 (Anderson et al., 1993, Biochem. Biophys. Res. Commun. 194, 876-884) and 49-58 (having the basic sequence RKKRRQRRR) of HIV-Tat. Vives et al. (1997), J Biol Chem 272, 16010-7 identified a sequence consisting of amino acids 48-60 (CGRKKRRQRRRPPQC), which appears to be important for translocation, nuclear localisation and trans-activation of cellular genes. Intraperitoneal injection of a fusion protein consisting of -galactosidase and a HIV-TAT protein transduction domain results in delivery of the biologically active fusion protein to all tissues in mice (Schwarze et al., 1999, Science 285, 1569-72)

The third helix of the Drosophila Antennapedia homeodomain protein has also been shown to possess similar properties (reviewed in Prochiantz, A., 1999, Ann NY Acad Sci, 886, 172-9). The domain responsible for translocation in Antennapedia has been localised to a 16 amino acid long peptide rich in basic amino acids having the sequence RQIKIWFQNRRMKWKK (Derossi, et al., 1994, J Biol Chem, 269, 10444-50). This peptide has been used to direct biologically active substances to the cytoplasm and nucleus of cells in culture (Theodore, et al., 1995, J. Neurosci 15, 7158-7167). Cell internalisation of the third helix of the Antennapedia homeodomain appears to be receptor-independent, and it has been suggested that the translocation process involves direct interactions with membrane phospholipids (Derossi et al., 1996, J Biol Chem, 271, 18188-93).

The VP22 tegument protein of herpes simplex virus is capable of intercellular transport, in which VP22 protein expressed in a subpopulation of cells spreads to other cells in the population (Elliot and O'Hare, 1997, Cell 88, 223-33). Fusion proteins consisting of GFP (Elliott and O'Hare, 1999, Gene Ther 6, 149-51), thymidine kinase protein (Dilber et al., 1999, Gene Ther 6, 12-21) or p53 (Phelan et al., 1998, Nat Biotechnol 16, 440-3) with VP22 have been targeted to cells in this manner.

Particular domains or sequences from proteins capable of translocation through the nuclear and/or plasma membranes may be identified by mutagenesis or deletion studies. Alternatively, synthetic or expressed peptides having candidate sequences may be linked to reporters and translocation assayed. For example, synthetic peptides may be conjugated to fluoroscein and translocation monitored by fluorescence microscopy by methods described in Vives et al. (1997), J Biol Chem 272, 16010-7. Alternatively, green fluorescent protein may be used as a reporter (Phelan et al., 1998, Nat Biotechnol 16, 440-3).

Any of the domains or sequences or as set out above or identified as having translocational activity may be used to direct the immunoglobulins into the cytoplasm or nucleus of a cell. The Antennapedia peptide described above, also known as penetratin, is preferred, as is HIV Tat. Translocation peptides may be fused N-terminal or C-terminal to single domain immunoglobulins according to the invention. N-terminal fusion is preferred.

Also of use for the delivery of antibodies to cells is the TLM peptide. The TLM peptide is derived from the Pre-S2 polypeptide of HBV. See Oess S, Hildt E Gene Ther 2000 May 7:750-8. Anti-DNA antibody technology is also of use. Anti-DNA antibody peptide technology is described in Alexandre Avrameas et al., PNAS val 95, pp 5601-5606, May 1998; Thérèse Ternynck et al., Journal of Autoimmunity (1998) 11, 511-521; and Bioconjugate Chemistry (1999), vol 10Number 1, pp 87-93.

Libraries According to the Invention

In a further aspect the present invention provides a library, wherein the library is generated from any one or more of the framework region sequences selected from the group shown in FIG. 3 consisting of: VH I21 (SEQ ID No: 4), VHI21R33 (SEQ ID No: 7), or either of those sequences wherein one or more of amino acids 22 and 92 are not cysteines; VLI21 (SEQ ID No:14) and VLI21R33 (SEQ ID No:17).

Systems, in which diverse peptide sequences are displayed on the surface of filamentous bacteriophage (Scott and Smith (1990 supra), have proven useful for creating libraries of antibody fragments (and the nucleotide sequences that encoding them) for the in vitro selection and amplification of specific antibody fragments that bind a target antigen. The nucleotide sequences encoding the V_{H} and V_{L} regions are inked to gene fragments which encode leader signals that direct them to the periplasmic space of E. coli and as a result the resultant antibody fragments are displayed on the surface of the bacteriophage, typically as fusions to bacteriophage coat proteins (e.g., pIII or pVIII). Alternatively, antibody fragments are displayed externally on lambda phage capsids (phagebodies). An advantage of phage-based display systems is that, because they are biological systems, selected library members can be amplified simply by growing the phage containing the selected library member in bacterial cells. Furthermore, since the nucleotide sequence that encode the polypeptide library member is contained on a phage or phagemid vector, sequencing, expression and subsequent genetic manipulation is relatively straightforward.

Methods for the construction of bacteriophage antibody display libraries and lambda phage expression libraries are well known in the art (McCafferty et al. (1990) supra; Kang et al. (1991) Proc. Natl. Acad Sci. USA., 88: 4363; Clackson et al. (1991) Nature, 352: 624; Lowman et al. (1991) Biochemistry, 30: 10832; Burton et al. (1991) Proc. Natl. Acad Sci U.S.A., 88:10134; Hoogenboom et al. (1991) Nucleic Acids Res., 19: 4133; Chang et al. (1991) J. Immunol., 147: 3610; Breitling et al. (1991) Gene, 104: 147; Marks et al. (1991) supra; Barbas et al. (1992) supra; Hawkins and Winter (1992) J. Immunol., 22: 867; Marks et al., 1992, J. Biol. Chem., 267: 16007; Lerner et al. (1992) Science, 258: 1313, incorporated herein by reference).

Alternative library selection technologies include bacteriophage lambda expression systems, which may be screened directly as bacteriophage plaques or as colonies of lysogens, both as previously described (Huse et al. (1989) Science, 246: 1275; Caton and Koprowski (1990) Proc. Natl. Acad Sci. U.S.A., 87; Mullinax et al. (1990) Proc. Natl. Acad. Sci. U.S.A., 87: 8095; Persson et al. (1991) Proc. Natl. Acad Sci. U.S.A., 88: 2432) and are of use in the invention. Whilst such expression systems can be used to screening up to 10⁶ different members of a library, they are not really suited to screening of larger numbers (greater than 106 members). Other screening systems rely, for example, on direct chemical synthesis of library members. One early method involves the synthesis of peptides on a set of pins or rods, such as described in WO84/03564. A similar method involving peptide synthesis on beads, which forms a peptide library in which each bead is an individual library member, is described in U.S. Pat. No. 4,631,211 and a related method is described in WO92/00091. A significant improvement of the bead-based methods involves tagging each bead with a unique identifier tag, such as an oligonucleotide, so as to facilitate identification of the amino acid sequence of each library member. These improved bead-based methods are described in WO93/06121.

Another chemical synthesis method involves the synthesis of arrays of peptides (or peptidomimetics) on a surface in a manner that places each distinct library member (e.g., unique peptide sequence) at a discrete, predefined location in the array. The identity of each library member is determined by its spatial location in the array. The locations in the array where binding interactions between a predetermined molecule (e.g., a receptor) and reactive library members occur is determined, thereby identifying the sequences of the reactive library members on the basis of spatial location. These methods are described in U.S. Pat. No. 5,143,854; WO90/15070 and WO92/10092; Fodor et al. (1991) Science, 251: 767; Dower and Fodor (1991) Ann. Rep. Med. Chem., 26: 271.

Other systems for generating libraries of polypeptides or nucleotides involve the use of cell-free enzymatic machinery for the in vitro synthesis of the library members. In one method, RNA molecules are selected by alternate rounds of selection against a target ligand and PCR amplification (Tuerk and Gold (1990) Science, 249: 505; Ellington and Szostak (1990) Nature, 346: 818). A similar technique may be used to identify DNA sequences which bind a predetermined human transcription factor (Thiesen and Bach (1990) Nucleic Acids Res., 18: 3203; Beaudry and Joyce (1992) Science, 257: 635; WO92/05258 and WO92/14843). In a similar way, in vitro translation can be used to synthesise polypeptides as a method for generating large libraries. These methods which generally comprise stabilised polysome complexes, are described further in WO88/08453, WO90/05785, WO90/07003, WO91/02076, WO91/05058, and WO92/02536. Alternative display systems which are not phage-based, such as those disclosed in WO95/22625 and WO95/11922 (Affymax) use the polysomes to display polypeptides for selection. These and all the foregoing documents also are incorporated herein by reference.

Practically the major advantage in the generation of libraries from the consensus sequences as herein described is that their production will obviate the use of phage scFv libraries and will reduce the necessary library size required for use in intracellular antibody capture technology.

Uses of Antibodies According to the Present Invention.

Antibody molecules according to the present invention, preferably scFv molecules may be employed in in vivo therapeutic and prophylactic applications, in vitro and in vivo diagnostic applications, in vitro assay and reagent applications, in functional genomics applications and the like.

Therapeutic and prophylactic uses of antibodies and compositions according to the invention involve the administration of the above to a recipient mammal, such as a human. Preferably they involve the administration to the intracellular environment of a mammal.

Substantially pure antibodies of at least 90 to 95% homogeneity are preferred for administration to a mammal, and 98 to 99% or more homogeneity is most preferred for pharmaceutical uses, especially when the mammal is a human. Once purified, partially or to homogeneity as desired, the antibody molecules may be used diagnostically or therapeutically (including extracorporeally) or in developing and performing assay procedures using methods known to those skilled in the art.

In the instant application, the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Animal model systems which can be used to screen the effectiveness of the selected antibodies of the present invention in protecting against or treating disease are available. Suitable models will be known to those skilled in the art.

Generally, the selected antibodies of the present invention will be utilised in purified form together with pharmacologically appropriate carriers. Typically, these carriers include aqueous or alcoholic/aqueous solutions, emulsions or suspensions, any including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The selected antibodies of the present invention may be used as separately administered compositions or in conjunction with other agents. These can include various immunotherapeutic drugs, such as cylcosporine, methotrexate, adriamycin or cisplatinum, and immunotoxins. Pharmaceutical compositions can include “cocktails” of various cytotoxic or other agents in conjunction with antibodies of the present invention or even combinations of the antibodies, according to the present invention.

The route of administration of pharmaceutical compositions according to the invention may be any of those commonly known to those of ordinary skill in the art. For therapy, including without limitation immunotherapy, the selected antibodies of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. The dosage and frequency of administration will depend on the age, sex and condition of the patient, concurrent administration of other drugs, counterindications and other parameters to be taken into account by the clinician.

The selected antibodies of the present invention can be lyophilised for storage and reconstituted in a suitable carrier prior to use. Known lyophilisation and reconstitution techniques can be employed. It will be appreciated by those skilled in the art that lyophilisation and reconstitution can lead to varying degrees of functional activity loss and that use levels may have to be adjusted upward to compensate.

The compositions containing the present selected antibodies of the present invention or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of selected immunoglobulin per kilogram of body weight, with doses of 0.05 to 2.0 mg/kg/dose being more commonly used. For prophylactic applications, compositions containing the present selected immunoglobulin molecules or cocktails thereof may also be administered in similar or slightly lower dosages.

A composition containing one or more selected antibody molecules according to the present invention may be utilised in prophylactic and therapeutic settings to aid in the alteration, inactivation, killing or removal of a select target cell population in a mammal. In addition, the selected repertoires of polypeptides described herein may be used extracorporeally or in vitro selectively to kill, deplete or otherwise effectively remove a target cell population from a heterogeneous collection of cells. Blood from a mammal may be combined extracorporeally with the selected antibodies, cell-surface receptors or binding proteins thereof whereby the undesired cells are killed or otherwise removed from the blood for return to the mammal in accordance with standard techniques.

The invention is further described, for the purposes of illustration only, in the following examples.

### EXAMPLES

### Example 1

Materials and Methods

Ras Antigen

Recombinant oncogenic HRAS (G12V; residues 1-166) was expressed in bacterial cells harbouring expression plasmids based on pET11a (Novagen) and purified by ion-exchange chromatography and gel-filtration described elsewhere (Pacold et al., 2000). To prepare the active form of RAS antigen, 3 mg of purified HRASG12V protein was loaded with 2 mM of 5′-guanylylimidodi-phosphate (GppNp, Sigma), non-hydrolysable analogue of GTP, using the alkaline phosphatase protocol (Herrmann et al., 1996). This GppNp-bound HRASG12V was used as antigen throughout.

In Vitro scFv Phage Library Screening and Preparation of Specific scFv-VP16 Yeast Library.

The IAC screening of three different scFv libraries (de Wildt et al., 2000; Sheets et al., 1998) were performed as described (Tse et al., 2000; Tse et al., 2002) (see also a link within the Laboratory of Molecular Biology website http://mrc-lmb.cam.ac.uk) with slight modifications. In outline, a first panning step used phage Ab library screens was performed using 50 μg/ml HRASG12V antigen in PBS containing 1 mM MgCl₂, bound to immunotubes. Anti-RAS bound phage were rescued and amplified in E. coli TG1. The scFv DNA fragments were sub-cloned into the pVP16 yeast vector and 4.13×10⁶ clones used for yeast screening. The RAS bait was prepared by cloning truncated HRASG12V cDNA into the EcoRI-BamHI site of pBTM116 vector. The pBTM116-RASG12 V bait vector (tryp+) was transfected into S. cerevisiae L40 using the lithium acetate/polyethylene glycol method (Tse et al., 2000), and colonies growing on Trp plates were selected. The expression of LexA-RAS fusion protein was confirmed by Western blot using anti-pan RAS (Ab-3, Oncogene Research Product). For library screening, 100 μg of yeast scFv-VP16 library DNA were transformed into L40 clone stably expressing antigen. Positive colonies were selected for His prototropy and confirmed by β-galactosidase (β-gal) activity by filter assay. For the isolated individual clones, false positive clones were eliminated and true positive clones were confirmed by re-testing of His independent growth and β-gal activation.

Purification of scFv for In Vitro Assay

Periplasmic bacterial expression of scFv was as described (Tse et al., 2000). The scFv were cloned into pHEN2 vector (see www.mrc-cpe.cam.ac.uk for map) and expressed for 2 hours at 30° C. with 1 mM ITPG in 1 litre culture of E. coli HB2151 cells. The cells were harvested and extracted periplasmic proteins with TES buffer (Tris-HCl (pH 7.5), EDTA, sucrose). The periplasmic proteins were dialyzed overnight against 2.5 litre of PBS including 10 mM imidazole. Immobilised metal ion affinity chromatograpy of periplasmic scFv was carried out at 4° C. for 1 hour with 4 ml of Ni-NTA agarose (QIAGEN). The agarose was washed 4 times with 20 ml of PBS with 20 mM imidazole. The polyhistidine-tagged scFv were eluted with 4 ml of 250 mM imidazole in PBS. The eluate was dialyzed overnight against 2.5 liter of 20 mM Tris-HCl (pH 7.5) including 10% glycerol at 4° C. Purified scFv was concentrated to 1 to 5 mg/ml using Centricon concentrator (YM-10, Amicon) and the aliquots were stored at −70° C. Protein concentration of purified scFv were measured using Bio-Rad Protein assay Kit (Bio-Rad).

ELISA Assays

The ELISA plate wells were coated with 10011 of purified HRASG12V-GppNp antigen (4 μg/ml, approximately 200 nM) in PBS overnight at 4° C. Wells were blocked with 3% bovine serum albumin (BSA)-PBS for 2 hours at room temperature. The respective purified scFv (approximately 450 ng) were diluted in 90 μl in 1% BSA-PBS and allowed to bind for 1 hour at 37° C. After washing 3 times with PBS containing 0.1% Tween-20 (PBST), horseradish peroxidase (HRP) conjugated anti-polyhistidine (HIS-1, Sigma) monoclonal antibody which were diluted 1:2000 in 1% BSA-PBS were allowed to bind for 1 hour at 37° C. After washing 6 times with PBST, HRP activity was visualised using 3,3′,5,5-tetramethylbenzidine (TMB) liquid substrate system according to manufacturer's instruction. The reaction was stopped with 0.5M hydrosulphate and data collected with a microtiter plate reader (450-650 nm filter). To verify the specificity of scFv with antigen, competitive ELISA assay was also performed. scFv were pre-incubated with HRASG12V-GppNp antigen (8 μg/ml) for 30 minutes at room temperature, before adding the mixture to the antigen coated ELISA wells. All measurements were performed in duplicate.

Surface Plasmon Resonance Analysis

The BIAcore 2000 (Pharmacia Biosensor) was used to measure the binding kinetics of scFv with antigen. To immobilise antigen on a CM5 sensorchip, the sensorchip was first activated by flowing 40 μl of the mixture of EDC/NHS(N-ethyl-N-(dimethylaminopropyl) carbodiimide hydrochloride/N-hydroxysuccinimide) at 10 μl/min flow rate. 100 μg/ml of purified HRASG12V-GppNp in 10 mM sodium acetate, pH 3.5 was injected and immobilised until approximately 1500 RU. After immobilisation, the chip was inactivated with 40 μl of ethanolamine-HCl. Purified scFv (10-500 nM) were loaded at flow rate of 201 μl/minute at 25° C. (running buffer HBS-EP (0.01 M HEPES, pH 7.4, 0.15M NaCl, 3 mM EDTA, 0.005% v/v polysorbate 20) plus 2 mM MgCl₂,) on 2 channels of the chip containing either immobilised HRASG12V-GppNp or no antigen, for the determination of the binding affinity of scFv. Each determination was performed in duplicate. The antigen immobilised surface on the sensorchip after binding scFv was regenerated by rinsing with 10 mM HCl until the starting baseline was achieved. The kinetic rate constants, kₒₙ and k_{off}, were evaluated using the BIAevaluation 2.1 software supplied by the manufacturer. Kd values were calculated from k_{off} and kₒₙ rate constants (Kd=k_{off}/kₒₙ).

Mammalian In Vivo Antigen-Antibody Interaction Assay.

The scFv were cloned into Sfi1 and Not1 site of pEF-BOS-VP16 expression vector (manuscript in preparation). The HRAS expression plasmid (pM1-RASG12V) expressing RASG12V in-frame with Gal4 DBD, was made by sub-cloning HRASG12V cDNA (codons 1-166) into EcoR1/BamH1 site of pM1 vector (Sadowski et al., 1992). The baits pM1/β-gal and the prey pEF-BOS-VP16/R4 (anti-β-gal scFv), used as positive or negative controls, have been described (Tse and Rabbitts, 2000). COS7 cells were transiently co-transfected with 500 ng of pG5-Luc reporter plasmid (de Wet et al., 1987), 50 ng of pRL-CMV (Promega), 500 ng of pEF-BOS-VP16/scFv and 500 ng of pM1/antigen bait with 8 μl of LipofectAMINE™ transfection reagent (Invitrogen, according to manufacture's instruction). Forty-eight hours after transfection, the cells were washed once with PBS and lysed in 500 μl of 1× passive lysis buffer (Promega) at room temperature for 15 min with gently shaking. 20 μl of cell lysate was assayed using Dual-Luciferase Reporter Assay System (Promega) in a luminometer. Transfection efficiency was normalised with the Renilla luciferase activity. The fold luciferase activity was calculated by dividing the normalised Firefly luciferase activity of the sample containing the vector alone. The data represent two experiments performed in duplicate.

Immunofluorescence Assays

scFv DNA fragments were cloned into the NcoI-NotI site of pEF-nuc-myc (Invitrogen with nuclear localisation signal (nls) at N-terminal and myc-tag at C-terminal of expressed scFv. For expression of RAS antigen, full length RASG12V cDNA was cloned into the Kpn1-EcoR1 site of pHM6 vector (Boehringer Mannheim) to encode RAS with HA-tag at N-terminal and His6-tag at C-terminal. The day before transfection, 1.2×10⁴ COS7 cells were seeded on Lab-Tek II Camber slide (Nalge Nunc International). The plasmids were co-transfected using Lipofectamine and forty-eight hours after transfection, cells were washed twice with PBS, permeabilised with 0.5% Triton X in PBS and fixed with 4% paraformaldehyde in PBS. Cells were stained with anti c-myc mouse monoclonal antibody (Santa Cruz; 9E10) and anti-HA rabbit polyclonal serum (Santa Cruz; sc-805) both at dilutions of 1:100. Secondary antibodies, fluorescein-linked sheep anti-mouse antibody and Cy3-linked goat anti-rabbit antibody (Amersham Pharmacia Biotech (APB)), were used at dilutions of 1:200 for staining. After several washes with PBS, the slides were overlaid with cover-slips and staining patterns were studied using a Bio-Radiance confocal microscope (Bio-Rad).

Western Blot Analysis

To evaluate the expression level and solubility of scFv in mammalian cells, the scFv or scFv-VP16 fusion proteins were expressed in COS7 cells. For scFv expression, scFv DNA fragments were cloned into Nco1/Not1 sites of pEF-myc-cyto expression vector (Invitrogen). The day before transfection, COS7 cells were seeded at about 2×10⁵ per well in 6-well culture plate (Nunc). 1 μg of pEF-myc-cyto-scFv or pEF-BOS-scFv-VP16 were transiently transfected with 8 μl of LipofectAMINE. 48 hours after transfection, the cells were washed once with PBS, lysed for 30 minutes in ice cold extraction buffer (1 mM HEPES, pH 7.6, 250 mM NaCl, 5 mM EDTA, 0.5% NP-40, 1 μg/ml leupeptin, 1 μg/ml pepstatin A, 0.1 mg/ml aprotinin, 1 mM phenylmethanesulsonyl fluoride (PMSF)) and centrifuged for 10 minutes at 13,000 rpm at 4° C. The pellets (“insoluble” fraction) and the supernatants (“soluble” fraction) were analysed by SDS-PAGE, followed by Western blot using anti-myc (9E10) monoclonal antibody (for detection of scFv) or anti-VP16 (14-5, Santa-Cruz) monoclonal antibody (for scFv-VP16AD fusion) as primary antibody and HRP-conjugated rabbit anti-mouse IgG antibody (APB) as secondary antibody. The blots were visualised by enhanced chemiluminescence (ECL) detection kit (ABP)

Mutation of Framework Residues for anti-RAS scFv.

pEF-BOS-VP16 with anti-RAS scFv33 was used for original templates and Pfu DNA polymerase was used throughout. The construct 12R33 (sequence shown in FIG. 3), which comprises FRs of anti-RAS scFvI21 and the CDRs of anti-RAS scFv33, was constructed using step-by-step site-specific mutagenesis of scFv33 as primary template using footprint mutagenesis. I21R33 (VHC22S;C92S), con33 and I21R33VHI21VL (FIG. 3) were also constructed by mutations of I21R33 using footprint mutagenesis with appropriate oligonucleotides. All scFv constructs were digested with Sfi1 or Nco1, and Not1 and subcloned into pEF-BOS-VP16 (for in vivo antigen antibody interaction assay) and pEF-myc-cyto vector (for expression of scFv in mammalian cells). All mutated scFv constructs were verified by DNA sequencing.

Transformation Assays in NIH3T3 Cells

RAS protein is localsied to the plasma membrane of cells and therefore to localise scFv to cell membrane we used the pEF-Memb vector (Invitrogen). The scFv expression plasmid was constructed by introducing carboxyl terminal 20 amino acid residues of HRAS into the Not1-Xba1 site of pEF-myc-cyto vector. This expression vector also was introduced FLAG-tag peptide (MDYKDDDDK) and alternative Sfi1 cloning site into blunt-ended Sfi1 site of pEF-Memb vector named pEF-FLAG-Memb. The scFv were sub-cloned into Sfi1-Not1 of pEF-FLAG-Memb. For expression of RASG12V, HRASG12V mutant cDNA were subcloned into expression vector pZIPneoSV(X) REF. Low passage NIH3T3 cells clone D4 (a gift from Dr Chris Marshall) were seeded at 2×10⁵ cells per well in 6-well plates the day before transfection, For transfection, 2 μg of each pEF-FLAG-Memb-scFv plus 100 ng of pZIPneoSV(X)-HRASG12V vector was used, using 12 μl of LipofectAMINE™. Two days later, the cells were transferred to 10 cm plates and grown for two weeks in DME medium containing 5% donor calf serum (Invitrogen) and penicillin and streptomycin. The plates were finally stained with crystal violet and the number of foci counted.

### Example 2

Isolation of Specific Intracellular Antibody Fragments which Recognise RAS Protein In Vivo

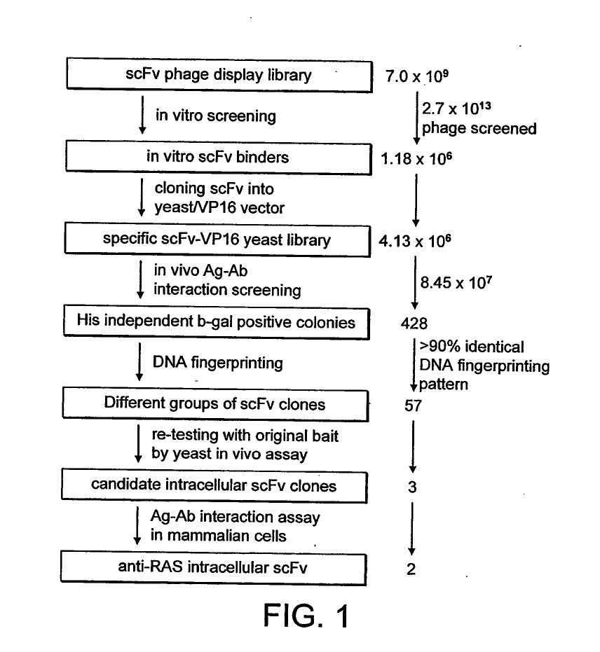

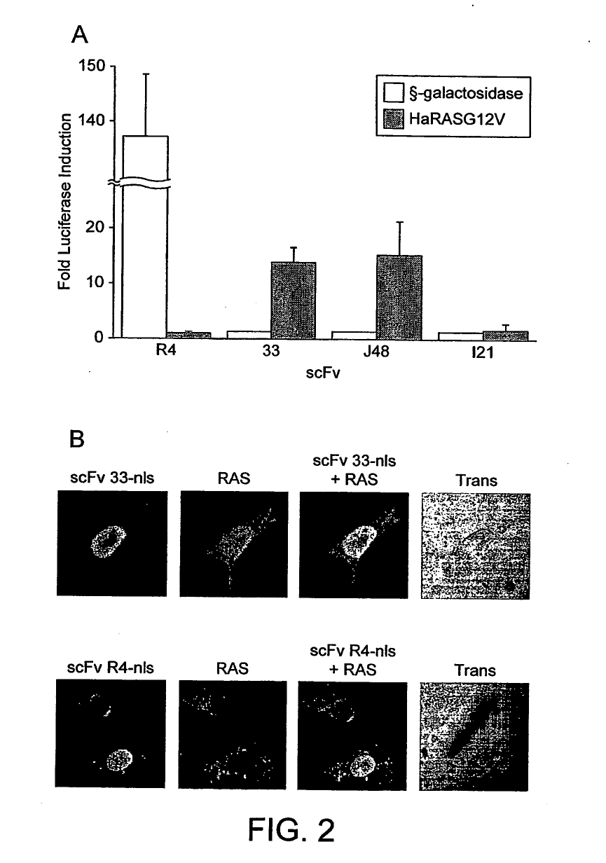

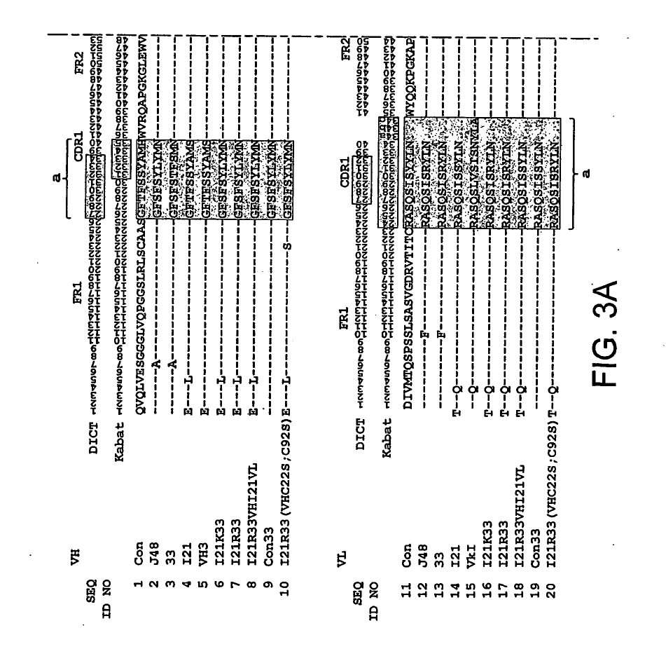

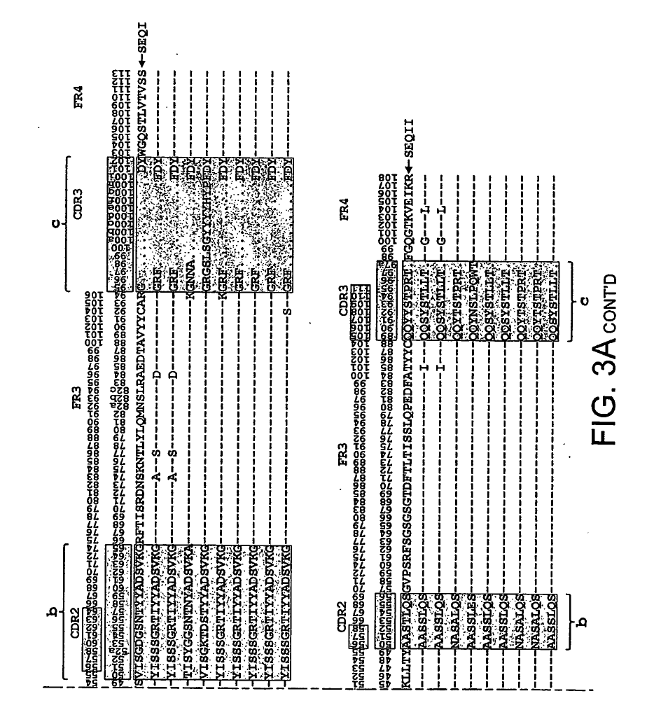

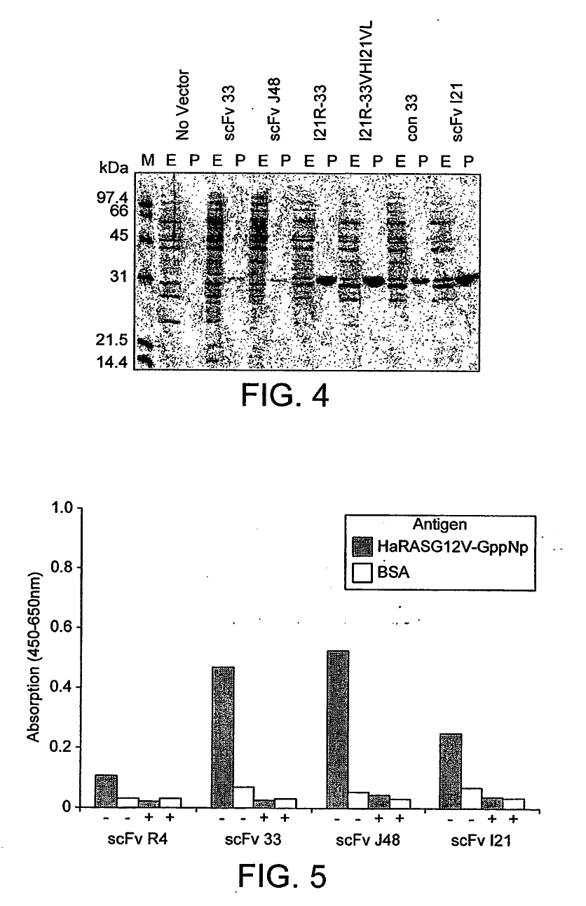

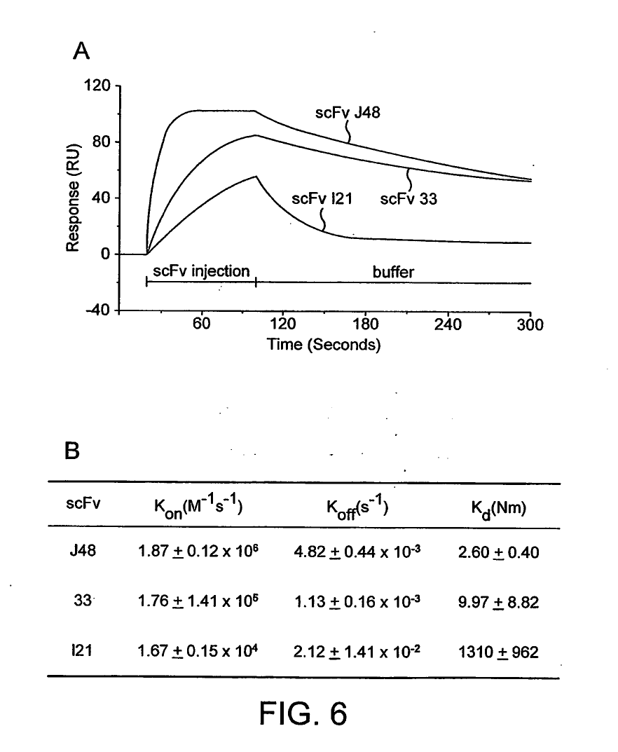

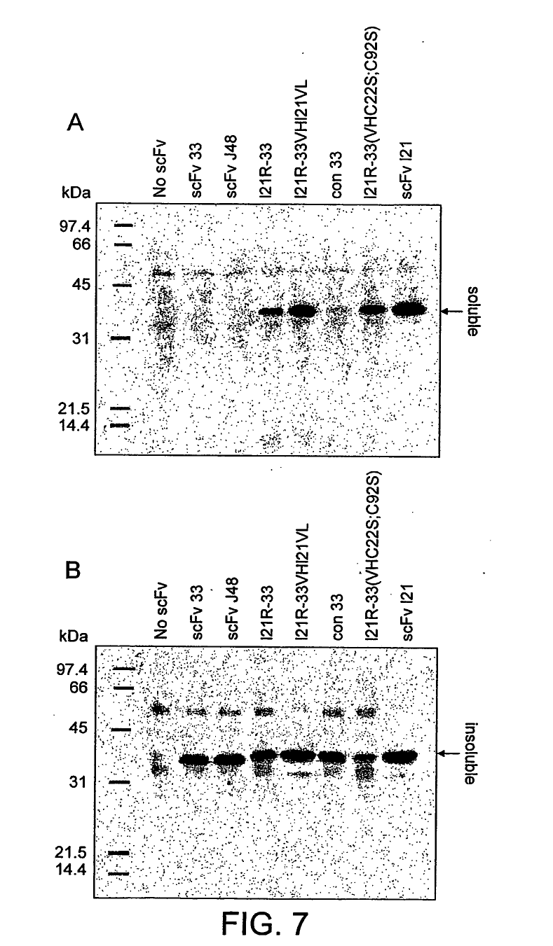

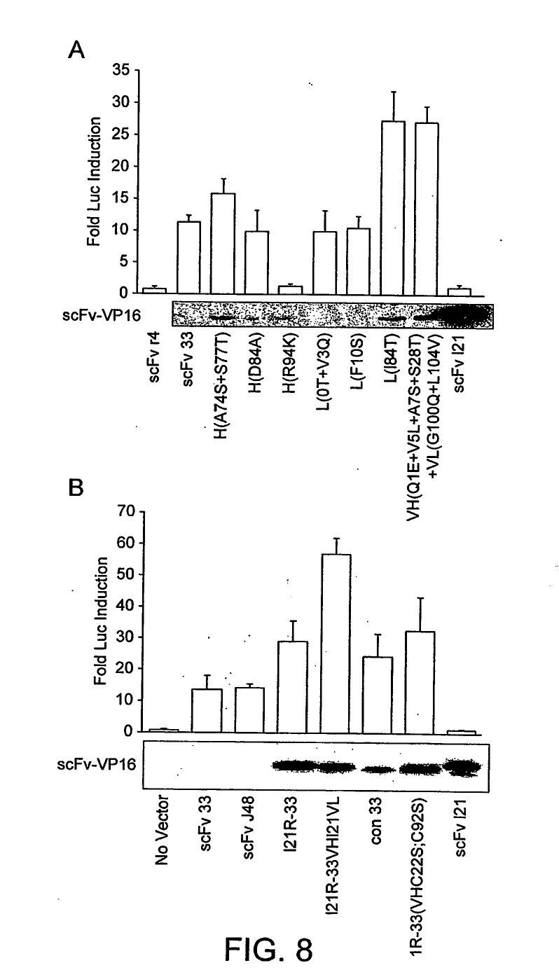

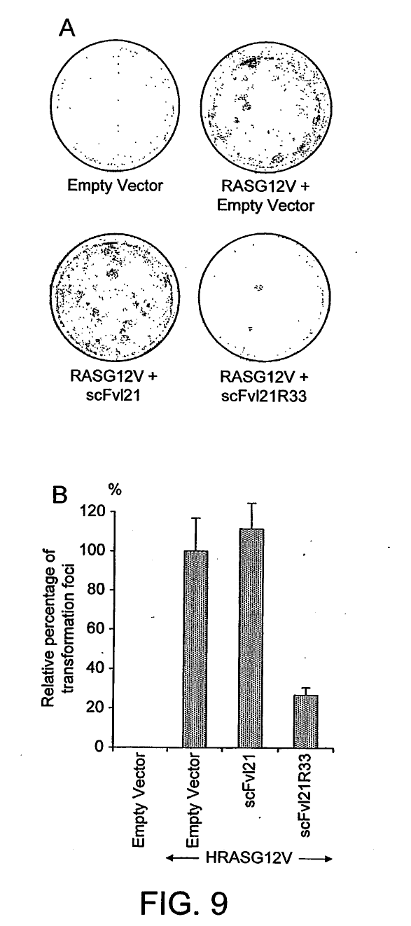

We have applied the intracellular antibody capture technique (Visintin et al., 1999) to the isolation of anti-RAS ICAbs. The sequential steps comprise initial in vitro phage scFv library panning with purified RAS protein and in vivo antigen-antibody two hybrid interaction screening to isolate specific intracellular antibodies. For in vitro phage Ab screen, purified carboxyl-terminal truncated human Ha-RASG12V was used as antigen, bound to 5′-guanylylimidodi-phosphate (GppNp, Sigma, non-hydrolysable analogue of GTP). After one round of in vitro panning, about 1.18×10⁶ antigen-bound phage were recovered from 2.7×10¹³ initial phage (FIG. 1). The sub-library was prepared as phagemid DNA and cloned into a yeast VP16 transcriptional activation domain vector to make an anti-RAS scFv-VP 16-AD library (about 4×10⁶ clones). This yeast sub-library was transfected into a yeast strain (L40 with his and -gal reporter genes) expressing the fusion protein bait comprising the LexA-DBD fused to RAS-G12V. A total of approximately 8.45×10⁷ yeast colonies were screened (FIG. 1). 428 colonies grew in the absence of histidine and these clones also showed activation of β-gal. The scFv-VP 16-AD plasmids were isolated from the histidine-independent, β-gal positive clones and assorted by their DNA restriction patterns. More than 90% of these scFv-VP 16-AD plasmids had an identical DNA finger printing pattern and twenty were sequenced and found to have identical DNA sequences. Those scFv with differing DNA finger print patterns were co-transformed with the pBTM/RASG12V bait in fresh yeast and assayed for histidine-independent growth and β-gal activation. Three anti-RAS scFv, designated 33, J48 and I21, were thus identified (FIG. 1). The specificity of these scFv for binding to RAS in yeast was further verified by their lack of interaction with the LexA DBD (made from the empty pBTM 116 vector) and a non-relevant antigen (&galactosidase) (data not shown).

The efficacy of the anti-RAS ICAbs was confirmed using a mammalian cell reporter assay and in vivo antigen co-location assays (FIG. 2). The mammalian cell assay used was luciferase production from a luciferase reporter gene. The three scFv were shuttled into a mammalian expression vector, pEF-BOS-VP16, which has the elongation factor-1a promoter (Mizushima and Nagata, 1990) and the VP 16 transcriptional activation domain (AD). The scFv were cloned in frame with the VP16 segment, on its N-terminal side (Triezenberg et al., 1988). The RASG12V antigen was cloned into the pM vector (Sadowski et al., 1992) which has the GAL4-DBD as an N-terminal fusion with antigen (pM-RASG12V). pEFBOSVP16-scFv and pM-RASG 12V were co-transfected into COS7 cells with the luciferase reporter plasmid. More than 10-fold activation was observed when scFv33 or J48 ICAb-VP16 fusion were expressed with the bait antigen RASG12V (FIG. 2A) but none with a non-relevant antigen β-galactosidase. No activation was observed, however, when the yeast anti-RAS ICAb I21 was co-expressed with RASG12V bait (FIG. 2A). Similar results were obtained in other mammalian cell lines viz. Hela and CHO cells. The failure of ICAb I21 to detectably interact with antigen in this mammalian cell assay, as opposed to yeast, may simply be due to it having insufficient affinity or may reflect the relative insensitivity of mammalian compared to yeast assays, perhaps due to factors such as transfection efficiency, reporter gene activation requiring access to endogenous transcription factors and/or the expression level of antigen or antibody.

The observed interaction of scFv33 and J48 in a yeast system expressing lexA-DBD and a mammalian system expressing Gal4-DBD is a good indicator that the scFv interact with a native epitope of the RAS antigen, rather than an artificial one due to fusion of RAS and a DBD in the bait. Additional evidence for this was obtained from co-location assays in which the native RAS antigen was expressed together with the scFv to which nuclear localisation signals (nls) had been added. COS7 cells were co-transfected with a RAS expression vector with HA epitope tag and scFv expression vectors encoding scFv with a myc epitope tag. After 52 hours, RAS antigen was detected with anti-HA tag Ab and scFv with anti-myc tag Ab (FIG. 2B). When the RAS antigen was expressed alone or with a non-relevant ICAb (scFvR4 (Martineau et al., 1998)), the antigen was detected in the cytoplasm and antibody in the nucleus (FIG. 2B, lower panels), whereas if the antigen was co-expressed with the anti-RAS ICAb 33 with a nls, co-location of RAS antigen and scFv was observed in the nucleus. These means that the anti-RAS ICAbs 33 have sufficient expression and affinity to bind RAS antigen in vivo and cause re-location within the cell (similar results were found with anti-RAS scFv J48, data not shown).

### Example 3

The Sequence and Bacterial Expression of anti-RAS scFv

The anti-RAS scFv (33, J48 and I21) were sequenced and derived protein sequence aligned (FIG. 3). All three scFv belong to VH3 subgroup joined to the JH5 and to the V 1 subgroup. Our previous data on anti-BCR and anti-ABL scFv (Tse et al., 2002), which were isolated only from the library of Sheets et al (Sheets et al., 1998), also belong to VH3 and V 1 subgroup. In our previous study we were able to define a consensus framework which was derived by comparing the anti-BCR and anti-ABL scFv (Tse et al., 2002) and an analogous study was conducted with anti-TAU ICAbs (Visintin et al., 2002). We concluded that a framework composed of VH3 and V 1 is highly amenable for scFv function inside the cell and the consensus set a basic sequence on which to design other ICAbs. The anti-RAS ICAbs described here help to refine this concept.

The levels of expression of three anti-RAS scFv were initially examined by bacterial periplasmic expression. These scFv were sub-cloned into pHEN2, which has the PelB leader sequence 5′ to the scFv, allowing the periplasmic expression of soluble scFv protein (see www.mrc-cpe.cam.ac.uk for map). Periplasmic scFv extracts were purified by immobilised metal ion affinity chromatography (IMAC) and protein preparations separated by SDS-PAGE (FIG. 4). The scFvI21 accumulated mainly in the soluble fraction, when secreted to the periplasm at 30° C. and the periplasmic expression yield was approximately 3 mg per litre culture. The other anti-RAS scFv (33 and J48) were expressed at less than 0.1 mg per litre. Comparison of the anti-RAS scFv sequences with the consensus ICAb sequence (FIG. 3), reveals only four differences in the VH framework residues of 33 and J48, one of which is position 7 in VH FR1. This residue is one of three which influence conformation of this region (Jung et al., 2001) and may thus influence ICAb 33 and J48 solubility. I21 conforms to the consensus in positions VH FR1 6, 7 and 10.

### Example 4

Biochemical and Biophysical Characterisation of anti-RAS scFv

The properties of the ICAbs isolated in our work were aslo characterised using two in vitro assays. The interaction of the scFv with RAS antigen was investigated with ELISA and biosensor assays using purified scFv made in bacteria RASG12V-GppNp was coated as antigen onto ELISA plates, challenged with purified scFv and bound scFv was detected using HRP conjugated anti-His tag antibody (FIG. 5). All three anti-RAS scFv produced significant signals with RAS antigen compared with BSA and the signals were inhibited by pre-incubation with RASG12V antigen, as a measure of specificity of the interaction. These results further suggest that these anti-RAS scFv may interact only with the native form RASG12V-GppNp.

The affinities of binding anti-RAS scFv to antigen were measured by binding kinetics in the BIAcore (FIG. 6). The Kd of scFv33 and J48 were determined to be 1.39 1.31 nM, 3.63±0.15 nM (FIG. 6B). The affinity difference of their scFv may reflect the differences of CDR1 sequence in VH domain. The scFvI21 had a Kd of 2.16±0.25 μM, about three order of magnitude weaker than scFv33 or J48. This weak affinity of scFvI21, in the micromolar range, is consistent with its weak β-gal reporter gene activation in the yeast in vivo antigen-antibody interaction assay and lack of detectable binding in mammalian cell assays.

### Example 5

The Functional Improvement of Anti-RAS scFv by Modification of the scFv Framework Sequences

There is an excellent quantitative correlation between stability and yield of scFv when expressed in bacterial cells and mammalian cells, in which scFvI21 showed a higher expression yield in bacteria (FIG. 4) and in mammalian cell cytoplasm (FIG. 7), compared with the other two anti-RAS scFv. While all scFv tested in COS7 expression showed significant amounts of ‘insoluble’ scFv (FIG. 7B), the best expression levels were apparent for scFvI21 (FIG. 7A). The ability to improve solubility and stability of anti-RAS scFv33 in vivo, as well as in vitro, was assessed by mutating the framework of scFv33, to include some or all of the 13 amino acid differences between it and scFvI21 (FIG. 3). When scFv33 was mutated in the VH FR regions to make it equivalent to I21 (but including arg at the end of FR3 rather than lys), excellent in vivo solubility was found (FIG. 7A, I21R-33). In addition, mutation of both Cys residues, needed for intra-chain disulphide bonds, to Ser (FIG. 7, scFv I21R-33(VHC22S;C92S) had only a small effect on soluble expression levels.

The in vivo interaction of the various mutants of the scFv was assessed in COS7 cells using the luciferase reporter assay (FIG. 8). FIG. 8A shows expression and luciferase reporter data of various modifications of the scFv33 framework compared to levels with scFv33 itself or scFvR4 (anti β-gal negative control, (Martineau et al., 1998)) and scFvI21 which does not give significant luciferase activity. One notable mutation of scFv33 is Arg94Lys (numbering according to Kabat et al (Kabat et al., 1991), position 106 according to IMGT, Lefranc et al (Lefranc and Lefranc, 2001)) which completely eliminated reporter response (FIG. 8A) even though the expression of this scFv-VP16 is increased compared with original scFv 33 (FIG. 8A). The arginine residue at position 94 is very close to the antigen binding site (CDR3 of heavy chain) and may be involved in interaction with RAS antigen directly. Alternatively, the residue at this position may form a surface bridge across the CDR3 loop through its positively charged side chain with the carboxyl group of the aspartic acid at position H101 (Morea et al., 1998), and the substitution (Arg to Lys) may affect the critical conformation of CDR3. The other mutant scFv33 variants generally maintained their binding ability with RAS antigen as judged by the luciferase reporter assay (FIG. 8A). Interestingly, three mutant variants, VH(A74S+S77T), VL(184T), and VH(Q1E+V5L+A7S+S28T)+VL(G100Q+L104V), were increased 1.5 to 2.5-fold in reporter gene activity, accompanied with an increase of scFv-VP16 in the soluble fraction.

The mutation of scFv33 into the framework of scFvI21 was performed, except arginine at position H94 was maintained (I21R33). Two further scFv33 variants were constructed, one in which scFv33 was converted to the ICAb consensus framework (con33) and one in which mutation of only the VH frameworks was carried out (I21R-33VHI21VL, FIG. 3). In the mammalian reporter assay, 2 to 3 fold-increase of reporter gene activity was observed with I21R33 and con33, but with dramatically improved solubility compared with original scFv33 (FIG. 8B). These data show that the consensus, or I21, framework are most suitable scaffold for intracellular antibody expression and furthermore ICAb function can be improved using these frameworks.

### Example 6

Activity of Anti-RAS scFv Lacking Conserved Cysteine Residues in VH Domain.

The mutated anti-RAS scFv I21R33 interacts specifically with RAS antigen in COS7 cells, even though, in this reducing environment, scFv mostly cannot form disulphide bonds (Biocca et al., 1995; Tavladoraki et al., 1993). Perhaps a small population of over-expressed scFv does form disulphide bonds in the cytoplasm and interact with antigen in vivo, such as the anti-β galactosidase scFvR4, some of which is disulphide bounded in cytoplasm of bacteria (Martineau et al., 1998). Thus a small population could be detectable in vivo using our antigen-antibody interaction assay, if the scFv has a high affinity with antigen. However, in vitro studies have demonstrated that some scFv can be made which are disulphide-free but fold correctly (Proba et al., 1998; Worn and Pluckthun, 1998a). Therefore, to test the requirement for intra-chain S—S bonds, an expression vector encoding a mutant scFv lacking the cysteine residues at position 22 and 92 (Kabat numbering or 23 and 104 in IMGT numbering) was constructed. This scFv, based on the I21R33 sequence, had the two cys codons were mutated to serine (clone I21R33(VHC22S;C92S). A vector encoding this protein was tested in our mammalian reporter assay (FIG. 8B). The scFv protein was expressed at high levels and roughly comparable with those of I21R33 and I21 and the ability to activate the luciferase reporter was similar to the 12R33 scFv. These results show that anti-RAS scFv I21R33 can fold adequately without intra-chain disulphide bond and function inside cells in this condition.

### Example 7

Conversion of ICAbs into Anti-RAS Reagents to Block Tumorigenic Transformation

In the experiments discussed above, we sought to improve on the effectiveness of anti-RAS ICAbs by mutational analysis of the VH and VL framework regions to make them equivalent to canonical IAC consensus (Tse et al., 2002). A further test of the utility of our pre-determined consensus frameworks was carried out by assessing the ability of anti-RAS sequences to inhibit oncogenic RAS transformation of NIH3T3 cells. We evaluated this by taking as a staring point the scFv the I21 clone which was isolated from the yeast screening (FIG. 1) using RAS as a bait but which does not have any significant activity in mammalian cells, despite being well expressed (FIG. 7). Mutatgenesis of the scFv33 to I21R33 (i.e. I21 framework with VH and VL CDRs of scFv33) gives a well expressed protein able to activate the luciferase reporter (FIG. 8B). We have used this in a competitive transformation assays in which NIH3T3 cells were transfected with a plasmid expressing activated RAS alone (RASG12V) to yield transformed, foci (non-contact inhibited colonies) which can grow in multilayers and show a swirling appearance of spindle-shaped cells (FIG. 9A, RASG12V+empty scFv vector). When the NIH3T3 cells were co-transfected with the RASG12V vector together with one expressing scFvI21, essentially no difference to control was observed (FIGS. 9A and B) in keeping with the observed lack of activation of the RAS-dependent luciferase reporter assays. On the other hand, when RASG12V was expressed with the mutated I21 clone, scFv21R33, the number of transformed foci reduced to 30% presumably due to interaction of the scFv with the RASG12V expressed protein and preventing its function. Thus the consensus scaffolds provide a basis for creation of functional scFv in our experiments.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry, molecular biology and biotechnology or related fields are intended to be within the scope of the following claims.
Adjei, A. A. (2001) Blocking oncogenic Ras signaling for cancer therapy. J Natl Cancer Inst, 93, 1062-74.
Biocca, S., Pierandrei-Amaldi P., Campioni, N. and Cattaneo, A. (1994) Intracellular immunization with cytosolic recombinant antibodies. Biotechnology, 12, 396-399.
Biocca, S., Pierandrei-Amaldi, P. and Cattaneo, A. (1993) Intracellular expression of anti-p21ras single chain Fv fragments inhibits meiotic maturation of xenopus oocytes. Biochem Biophys Res Commun, 197,422-7.
Biocca, S., Ruberti F., Tafani, M., Pierandrei-Amaldi P. and Cattaneo, A. (1995) Redox state of single chain Fv fragments targeted to the endoplasmic reticulum, cytosol and mitochondria. Biotechnology (NY), 13, 1110-5.
Bird, R. E., Hardman, K. D., Jacobson, J. W., Johnson, S., Kaufman, B. M., Lee, S.-M., Lee, T., Pope, S. H., Riordan, G. S. and Whitlow, M. (1988) Single-chain antigen-binding proteins. Science, 42, 423-426.
Cattaneo, A. and Biocca, S. (1999a) The selection of intracellular antibodies. Trends Biotech.
Cattaneo, A. and Biocca, S. (1999b) The selection of intracellular antibodies. Trends Biotechnol, 17, 115-21.
Cepko, C. L., Roberts, B. E. and Mulligan, R. C. (1984) Construction and applications of a highly transmissible murine retrovirus shuttle vector. Cell, 37, 1053-62.
Cohen, P. A. (2002) Intrabodies. Targeting scFv expression to eukaryotic intracellular compartments. Methods Mol. Biol., 178, 367-378.
de Wet, J. R., Wood, K. V., DeLuca, M., Helinski, D. R. and Subramani, S. (1987) Firefly luciferase gene: structure and expression in mammalian cells. Mol Cell Biol, 7, 725-37.
de Wildt, R. M., Mundy, C. R., Gorick, B. D. and Tomlinson, I. M. (2000) Antibody arrays for high-throughput screening of antibody-antigen interactions. Nat Biotechnol, 18, 989-94.
Herrmann, C., Horn, G., Spaargaren, M. and Wittinghofer, A. (1996) Differential interaction of the ras family GTP-binding proteins H-Ras, Rap1A, and R-Ras with the putative effector molecules Raf kinase and Ral-guanine nucleotide exchange factor. J Biol Chem, 271, 6794-800.
Hollenberg, S. M., Sternglanz, R., Cheng, P. F. and Weintraub, H. (1995) Identification of a new family of tissue-specific basic helix-loop-helix proteins with a two-hybrid system. Molecular and Cellular Biology, 15, 3813-3822.
Huston, J. S., Levinson, D., Mudgett-Hunter, M., Tai, M.-S., Novoty, J., Margolies, M. N., Ridge, R. J., Bruccoleri, R. E., Haber, E., Crea, R and Oppermann, H. (1988) Protein engineering of antibody binding sites: Recovery of specific activity in an anti-digoxin single-chain Fv analogue produced in Escherichia coli Proc. Natl. Acad. Sci. USA, 85, 5879-5883.
Jung, S., Spinelli S., Schimmele, B., Honegger, A., Pugliese, L., Cambillau, C. and Pluckthun, A. (2001) The importance of framework residues H6, H7 and H10 in antibody heavy chains: experimental evidence for a new structural subclassification of antibody V(H) domains. J Mol Biol, 309, 701-16.
Kabat, E. A., Wu, T. T., Perry, H. M., Gottesman, K. S. and Foeller, C. (1991) Sequences of proteins of immunological interest. National Institutes of Health, Bethesda.
Lefranc, M.-P. and Lefranc, G. (2001) The Immunoglobulin Factsbook, Academic Press, London.
Lowy, D. R. and Willumsen, B. M. (1993) Function and regulation of ras. Annu Rev Biochem, 62, 851-91.
Marasco, W. A., Haseltine, B. A. and Chen, S. (1993) Design, intracellular expression, and activity of a human anti-human immunodeficient virus type 1 gp120 single-chain antibody. Proc. Natl. Acad. Sci. USA, 90, 7899-7893.
Martineau, P., Jones, P. and Winter, G. (1998) Expression of an antibody fragment at high levels in the bacterial cytoplasm. J Mol Biol, 280, 117-127.
Mizushima, S. and Nagata, S. (1990) pEF-BOS, a powerful mammalian expression vector. Nucl. Acids Res., 18, 5322.
Morea, V., Tramontano, A., Rustici, M., Chothia, C. and Lesk, A. M. (1998) Conformations of the third hypervariable region in the VH domain of immunoglobulins. J Mol Biol, 275, 269-94.
Pacold, M. E. Suire, S., Perisic, O., Lara-Gonzalez, S., Davis, C. T., Walker, E. H., Hawkins, P. T., Stephens, L., Eccleston, J. F. and Williams, R. L. (2000) Crystal structure and functional analysis of Ras binding to its effector phosphoinositide 3-kinase gamma. Cell, 103, 931-43.
Proba, K, Worn, A., Honegger, A. and Pluckthun, A. (1998) Antibody scFv fragments without disulfide bonds made by molecular evolution. J Mol Biol, 275, 245-53.
Rabbitts, T. H. (1994) Chromosomal translocations in human cancer. Nature, 372, 143-149.
Rabbitts, T. H. and Stocks, M. R. (2002) Chromosomal translocation products engender novel intracellular therapeutic technologies. submitted.
Rondon, I. J. and Marasco, W. A. (1997) Intracellular antibodies (antibodies) for gene therapy of infectious diseases. Annu Rev Microbiol, 51, 257-83.
Sadowski, I., Bell, B., Broad, P. and Hollis, M. (1992) GAL4 fusion vectors for expression in yeast or mammalian cells. Gene, 118, 137-141.
Sheets, M. D., Amersdorfer, P., Finnern, R., Sargent, P., Lindqvist, E., Schier, R., Hemingsen, G., Wong, C., Gerhart, J. C. and Marls, J. D. (1998) Efficient construction of a large nonimmune phage antibody library: the production of high-affinity human single-chain antibodies to protein antigens. Proc. Natl. Acad. Sci. USA, 95, 6157-6162.
Tanaka, T., Chung, G. T. Y., Forster, A., Lobato, M. N. and Rabbitts, T. H. (2002) De novo synthesis of antibody gene fragments and diversification by footprint mutagenesis. in prep.
Tavladoraki, P., Benvenuto, E., Trinca, S., De Martinis, D., Cattaneo, A. and Galeffi P. (1993) Transgenic plants expressing a functional single-chain Fv antibody are specifically protected from virus attack. Nature, 366, 469-472.
Triezenberg, S. J., Kingsbury, R. C. and McKnight, S. L. (1988) Functional dissection of VP16, the trans-activator of herpes simplex virus immediate early gene expression. Genes Dev, 2, 718-29.
Tse, E., Chung, G. and Rabbitts, T. H. (2000) Protocols for isolating antigen-specific intracellular antibody fragments as sinlge chain Fv for use in mammalian cells. In Walker, J. and Turksen, K. (eds.), Methods in Molecular Biology and Medicine. Humana Press, Totawa, Vol. in press.
Tse, E., Lobato, M. N., Forster, A., Tanaka, T., Chung, T. Y. G. and Rabbitts, T. H. (2002) Intracellular antibody capture technology: application to selection of single chain Fv recognising the BCR-ABL oncogenic protein. J. Mol. Biol., 317, 85-94.
Tse, E. and Rabbitts, T. H. (2000) Intracellular antibody-caspase mediated cell killing: a novel approach for application in cancer therapy. Proc. Natl. Acad. Sci. USA, 97, 12266-12271.
Visintin, M., Settanni, G., Maritan, A., Graziosi, S., Marks, J. D. and Cattaneo, A. (2002) The intracellular antibody capture technology (IACT): towards a consensus sequence for intracellular antibodies. J. Mol. Biol., 317, 73-83.
Worn, A. and Pluckthun, A. (1998a) An intrinsically stable antibody ScFv fragment can tolerate the loss of both disulfide bonds and fold correctly. Febs Lett, 427, 357-361.
Worn, A. and Pluckthun, A. (1998b) Mutual stabilization of VL and VH in single-chain antibody fragments, investigated with mutants engineered for stability. Biochemistry, 37, 13120-7.

1. A method for the generation of an antibody which is suitable for intracellular use, the method comprising the steps of:
(a) testing two or more antibodies for their ability to bind to one or more antigens specifically within an intracellular environment, and selecting an antibody which is capable of binding to one or more antigens specifically within such an environment.
(b) testing two or more antibodies for their ability to be soluble within an intracellular environment, and selecting an antibody which is soluble within such an environment; and
(c) generating said antibody suitable for intracellular use from the framework regions of the antibody selected in step (b) and the CDR sequences from the antibody selected in step (a).

2. A method for generating an antibody which is suitable for intracellular use, the method comprising the step of synthesising said antibody using at least a framework sequence or a nucleic acid sequence encoding it, selected from the group consisting of: the heavy chain framework region depicted in FIG. 3 as I21 and designated SEQ ID No: 4, the heavy chain framework region of the consensus sequence designated SEQ ID No: 1 and depicted as Con, the heavy chain framework region depicted in FIG. 3 as I21R33 and designated SEQ ID No: 7, the light chain framework region depicted in FIG. 3 as I21 and designated SEQ ID No: 14, the light chain framework region of the consensus sequence designated SEQ ID No: 11 and depicted as Con, the light chain framework region depicted in FIG. 3 as I21R33 and designated SEQ ID No: 17 or any of those heavy framework sequences wherein the amino acid residues at positions 22 and 92, according to the Kabat numbering system, are not cysteine residues.

3. A method for the synthesis of an antibody molecule which is suitable for intracellular use, the method comprising the step of synthesising the antibody from a heavy chain variable domain, or a nucleic acid encoding it, which heavy chain variable domain falls within the VHIII subgroup and which comprises one or more amino acids at the positions indicated which are selected from the group consisting of the following, according to Kabat numbering: VH position 1, glutamine; VH position 5 lysine; VH position 7, serine; VH position 74, serine; VH position 77, threonine; VH position 84, VH alanine, VH position 22, any amino acid other than cysteine; VH position 92, any other amino acid other than cysteine; and/or synthesising the antibody from a light chain variable domain which falls within the V_{K}1 subgroup and which comprises one or more amino acids at the positions indicated which are selected from the group consisting of the following, according to Kabat numbering: VL position 0, threonine; VL position 4, Glutamine.

4. A method for converting an antibody molecule which comprises a heavy chain variable domain which falls within the VHIII subgroup of heavy chains, into one which is suitable for intracellular use, the method comprising the step of mutating one or more framework amino acids comprised by at least one of the antibody's heavy chain variable domains to an amino acid, according to Kabat numbering, selected from the group consisting of: VH position 1, glutamine; VH position 5 lysine; VH position 7, serine; VH position 74, serine; VH position 77, threonine; VH position 84, VH alanine, VH position 22, any amino acid other than cysteine; VH position 92, any other amino acid other than cysteine.

5. A method for converting an antibody molecule which comprises a light chain variable domain which falls within the VK1 subgroup of light chains, into one which is which is suitable for intracellular use, the method comprising the step of mutating one or more framework amino acids comprised by at least one of the antibody's light chain variable domains to an amino acid, according to Kabat numbering, selected from the group consisting of: VL position 0, threonine; VL position 4, Glutamine.

6. A method for converting an antibody molecule into one which is suitable for intracellular use, the method comprising the step of mutating one or more framework amino acids comprised by at least the antibody's heavy chain variable domain so that the resultant framework amino acid sequence comprises a sequences selected from the group consisting of the following as shown in FIG. 3: SEQ ID No: 1 (VH-Con), SEQ ID No: 4 (VH-I21), SEQ ID No: 7 (VH-I21R33) 17; or any of those heavy framework sequences wherein the amino acid residues at positions 22 and 92, according to the Kabat numbering system, are not cysteine residues.

7. An antibody molecule obtained by the method of any one of claims 1-6.

8. An antibody molecule according to claim 7 which is a Dab.

9. An antibody molecule according to claim 7 which comprises both heavy and light chain variable domains.

10. A polypeptide library, wherein the library is generated from any one or more of the framework region sequences selected from the group shown in FIG. 3 consisting of: VHI21 (SEQ ID No: 4), VHI21R33 (SEQ ID No: 7), or either of those sequences wherein one or more of amino acids 22 and 92 are not cysteines; VL21 (SEQ ID No: 14) and VLI21R33 (SEQ ID No: 17).

11. A composition comprising an antibody according to claim 7 and a pharmaceutically acceptable carrier, diluent or exipient.

12. A method for the treatment or prophylaxis of a disease, the method comprising administering an antibody of claim 7 to an individual in need of such treatment or prophylaxis.

13. A method for specifically binding an antigen within an intracellular environment, the method comprising contacting an antibody of claim 7 with a said antigen wherein said contacting occurs in an intracellular environment.
